(19) United States
(12) Patent Application Publication
(10) Pub. No.: US 2009/0047338 A1
(43) Pub. Date: Feb. 19, 2009

(54) Method to Treat Flavivirus Infection with siRNA

(75) Inventors: Manjunath N. Swamy, Roslindale, MA (US); Premlata Shankar, Roslindale, MA (US); Priti Kumar, Boston, MA (US); Sang-Kyung Lee, Seoul (KR)

Correspondence Address: DAVID S. RESNICK, NIXON PEABODY LLP, 100 SUMMER STREET, BOSTON, MA, 02110-2131, US

(73) Assignee: IMMUNE DISEASE INSTITUTE, INC., Boston, MA (US)

(21) Appl. No.: 12/089,015

(22) PCT Filed: Oct. 5, 2006
(86) PCT No.: PCT/US2006/038980

§ 371 Date: Apr. 2, 2008

Related U.S. Application Data
(60) Provisional application No. 60/723,686, filed on Oct. 5, 2005.

(51) Int. Cl. A61K 9/127; A61K 31/7105; A61P 31/00

(52) U.S. Cl. 424/450; 514/44

(57) ABSTRACT

The present invention is directed to methods of treating flavivirus mediated diseases using siRNAs. The invention is based upon our findings in a mouse model that siRNAs directed against sequences conserved among multiple flaviviruses prevents and treats flavivirus infections. Accordingly, the present invention provides an isolated siRNA comprising a sense RNA and an antisense RNA strand or a single strand. The sense and the antisense RNA strands, or the single RNA strand, form an RNA duplex, and wherein the RNA strand comprises a nucleotide sequence identical to a target sequence of about 15 to about 30 contiguous nucleotides in flavivirus mRNA or mutant or variant thereof.

### CROSS-REFERENCE TO RELATED APPLICATIONS

This application claims benefit under 35 U.S.C. 119(e) of U.S. application Ser. No. 60/723,868, filed Oct. 5, 2005, the content of which is herein incorporated by reference in its entirety.

### GOVERNMENT SUPPORT

This invention was supported by the National Institutes of Health (NIH)/National Institute of Allergy and Infectious Disease (NIAID) Grant No. U19 A1056900, the Govemment of the United States has certain rights thereto.

### BACKGROUND OF THE INVENTION

The prototypical flavivirus, yellow fever virus (YFV), was first isolated in 1927. Since that time, the membership of the genus Flavivirus has grown to over 70 known viruses, of which more than half are associated with human disease. The flaviviruses have been sub-classified on the basis of antigenic relatedness, or more recently, on sequence similarity. Sequence information has been used to classify the viruses into 14 clades, which correlate closely with the previous antigenic classifications (Kuno, et al., J Virol. 72:73-83, 1998). The majority of the flaviviruses are vector borne, with approximately 50% transmitted by mosquitoes, and 30% carried by ticks. The remaining 20% are classified as “non-vector,” which are transmitted by an as yet unidentified vector or zoonotically from rodents or bats (e.g., see 2001 review by Burke & Monath, Flaviviruses, p. 1043-1125; in D. M. Knipe, and P. M. Howley (eds), Knipe, D. M. Howley, P. M., Fourth ed. Lippincott Williams & Wilkins, Philadelphia). The general transmission cycle of the vector borne viruses involves the acquisition of the virus by the arthropod through feeding on an infected host, typically birds, small mammals, or primates. The virus replicates in the insect host, which in turn can infect an immunologically naive bird (or small mammal or primate, depending on the virus).

In the case of WNV, human infection through the bite of an infected mosquito results in fever in 20 percent of cases, and 1 in 150 infections result in neurological disease. The greatest risk factor for neurological disease following infection appears to be advanced age. Most JEV infections are sub-clinical, with only 1 in 250 infections resulting in symptoms. The primary clinical manifestation is encephalitis. Symptoms begin with headache, fever, and gastrointestinal problems after a 5-15 day incubation period. These symptoms may be followed by irritability, nausea, and diarrhea with decline to generalized weakness, stupor, or coma. In children, seizures are common, and 5-30% of such cases are fatal. Recent reports show efficacy of ribavirin and interferon-alpha2b in WNV infection, although controlled clinical trials have not been completed (Petersen & Martin, Ann Intern Med. 137:173-9, 2002). Significantly, there is no specific treatment for WNV or JEV, other than supportive care. Treatment for most flavivirus infections resulting in disease includes fluid management, mechanical ventilation, and transfusion in case of severe hemorrhage.

Preventive vaccine strategies based on live, attenuated strains as well as the construction of chimeric viruses based on the backbones of approved flavivirus vaccines are being developed against WNV, Dengue, and others (Monath, T. P., Ann NY Acad Sci. 95 1: 1-12, 2001). Preventative vaccines do exist for JEV, including a formalin-inactivated vaccine, as well as a live attenuated strain. The inactivated version has been used widely in Japan and China since the 1960s, and is also licensed for use in the U.S. and Europe for those traveling to areas in which JEV is endemic. The attenuated virus has also seen wide use in China. Both vaccines, when delivered with appropriate booster regimens, have shown efficacies greater than 90% (Burke & Monath, supra; Tsai, et al., 1999, Japanese Encephalitis Vaccines, p. 672-710; in S. A. Plotkin, and W. A. Orenstein (eds), Vaccines. W.B. Saunders Company, Philadelphia). A vaccine consisting of formalin-inactivated WNV is approved for veterinary use in horses. However, using live and/or attenuated viruses pose a significant health risk to individuals with compromised immune systems, such as children, elderly individuals and individuals with illnesses, such as HIV-AIDS, autoimmune diseases and the like, who are also among the most vulnerable to have severe symptoms when affected with a flavivirus infection.

Therefore, there is a pronounced need in the art for novel therapeutic methods and compositions having utility for preventing or inhibiting flavivirus infection, and for treatment and/or prevention of conditions related to flavivirus infection.

### SUMMARY OF THE INVENTION

The present invention is directed to methods of treating flavivirus mediated diseases using siRNAs. The invention is based upon our findings in a mouse model that siRNAs directed against sequences conserved among multiple flaviviruses prevents and treats flavivirus infections. Accordingly, the present invention provides an isolated siRNA comprising a sense RNA and an antisense RNA strand or a single strand. The sense and the antisense RNA strands, or the single RNA strand, form an RNA duplex, and wherein the RNA strand comprises a nucleotide sequence identical to a target sequence of about 15 to about 30 contiguous nucleotides in flavivirus mRNA or mutant or variant thereof. In one embodiment, the virus is selected from the group consisting of Cacipacore virus, Koutango virus, Murray Valley encephalitis virus, St. Louis Encephalitis virus, Alfuy virus, Kunjin virus, Yaounde virus, West Nile virus, Japanese Encephalitis virus, Dengue virus or any combination thereof. In one embodiment, the virus selected from the group consisting of West Nile virus, Japanese Encephalitis virus, Dengue virus or any combination thereof.

In one embodiment, the siRNA is formulated with a pharmaceutically acceptable carrier to form an antiviral composition that can treat or prevent viral infection. In one embodiment, the viral infection is mediated by a virus selected from the group consisting of Cacipacore virus, Koutango virus, Murray Valley encephalitis virus, St. Louis Encephalitis virus, Alfuy virus, Kunjin virus, Yaounde virus, West Nile virus, Japanese Encephalitis virus, Dengue virus or any combination thereof. In one embodiment, the viral infection is mediated by a virus selected from the group consisting of West Nile virus, Japanese Encephalitis virus, Dengue virus or any combination thereof. In one embodiment, the target sequence is conserved between 2, 3, 4, 5, 6, 7, 8, 9 or 10 species of flavivirus. In one embodiment, the target is selected from a group consisting of capsid encoding gene, envelope encoding gene, non-structural protein 3 encoding gene, untranslated regions and any combination thereof. In one embodiment, the capsid encoding gene is not targeted. In another embodiment, the capsid encoding gene is targeted in combination with the envelope encoding gene, non-structural protein 3 encoding gene, untranslated regions and any combination thereof.

In another embodiment, the siRNA is administered in combination with a pharmaceutical agent for treating, alleviating symptoms relating to, and/or preventing infection secondary to flavivirus disease, which pharmaceutical agent is different from the siRNA and is selected from the group consisting of anticonvulsants, antinausea medicants, antibiotics for prevention of pneumonia and/or urinary tract infection or any combination thereof.

In another embodiment, the present invention provides a method of inhibiting expression of viral mRNA, or mutant or variant thereof, or preventing or treating viral mediated disease. In one embodiment, the virus mediating the disease is selected from the group consisting of Cacipacore virus, Koutango virus, Murray Valley encephalitis virus, St. Louis Encephalitis virus, Alfuy virus, Kunjin virus, Yaounde virus, West Nile virus, Japanese Encephalitis virus, Dengue virus or any combination thereof. In one embodiment, the virus mediating the disease is selected from the group consisting of West Nile virus, Japanese Encephalitis virus, Dengue virus or any combination thereof. The method comprises administering to a subject an effective amount of siRNA of the invention.

### BRIEF DESCRIPTION OF THE DRAWINGS

FIGS. 1A-1D show lentiviral delivery of FvE^{J} shRNA suppresses JEV replication in cell lines. FIG. 1A shows RNA from BHK21 cells stably transduced with VSV-G-pseudotyped shFvE^{J} or shLuc lentivirus that was probed with ³²P end-labeled synthetic FvE^{J} siRNA sense strand to detect intracellular processing of shRNA. Antisense strand of the synthetic FvE^{J} siRNA (siFvE^{J}) was used as positive control. Before loading, samples were normalized for total RNA content. FIG. 1B shows mock or lentivirally transduced BHK21 cells that were challenged with JEV at a multiplicity of infection (moi) of 10 and the viral replication monitored 60 h later by flow cytometry after staining the cells with a JEV envelope-specific antibody. Percent-infected cells are indicated. The results are representative of at least 3 independent experiments. FIG. 1C shows total RNA obtained from the control shLuc- or shFvE^{J} lentivirus-transduced BHK21 cells that were either uninfected (UI) or infected with JEV that was probed with JEV- or β-actin cDNA in Northern blot analysis. FIG. 1D shows BHK21 and Neuro 2a cells transduced with shFvE^{J}, pseudotyped with either VSV-G or RV-G were examined for GFP expression by fluorescence microscopy. Bright field images are shown as insets.

FIGS. 2A-F show shFvE^{J} protects mice against JEV-induced encephalitis. FIGS. 2A and 2B show Balb/c mice (10/group) were injected on days −4 and −2 ic with 2×10⁵ TU (FIG. 2A) or 2×10³ TU (FIG. 2B) of either shFvE^{J} or shLuc, pseudotyped with either VSV-G or RV-G. On day 0, they were injected at the same spot ic with 4 LD₅₀ of JEV 30 minutes after the third dose of lentivirus and the mice monitored for survival over time. FIG. 2C shows representative photomicrographs of hematoxylin & eosin stained horizontal brain sections obtained from mice treated with shFvE^{J} or shLuc lentivirus and infected with JEV for 5 days are shown. Magnifications are indicated. FIG. 2D shows mice were injected with lentiviruses and challenged with JEV as in FIG. 2A, and their brain homogenates, obtained 5 days after JEV challenge were plaque titrated on BHK21 cell monolayers. For shFvE^{J} lentivirus, viral titers after a single (1×) as well as 3 (3×) administrations are shown. The viral titers are shown as log pfu/total brain. Each symbol represents an individual mouse. FIG. 2E shows brain homogenates in FIG. 2D were pooled and 1, 10 or 50 μl of pooled homogenate inoculated onto Neuro 2a cells and the viral replication monitored by flow cytometry 5 days later. FIG. 2F shows mice (5/group) were injected ic with 2×10⁵ TU of shLuc or shFvE^{J}, challenged 30 min later with JEV at the indicated challenge doses of virus and observed for survival over time.

FIGS. 3A-3 show that FvE^{J} synthetic siRNA also protects against fatal JEV infection. FIG. 3A shows that transfection of Neuro 2a cells with i-Fect complexed siFvEJ confers protection against JEV infection comparable to lipofectamine transfection. Neuro 2a cells were transfected with siRNA mixed with i-Fect or lipofectamine and after 2 d, they were challenged with JEV at a MOI of ten. Viral replication was monitored 72 h postinfection by flow cytometry. Also shown is an overlay histogram of uninfected Neuro 2a cells and JEV-infected Neuro 2a cells treated prior to infection with either i-Fect/siLuc, lipofectamine/siFvEJ, or i-Fect/siFvEJ as indicated. FIG. 3B shows that i-Fect-complexed siFvEJ protects mice from JEV infection when injected 30 min but not 6 h after infection. Mice (five per group) were injected IC with four LD50 of JEV, and after 30 min or 6 h they were also injected at the same spot with 0.5 nmoles of either siLuc or siFvEJ complexed with i-Fect and monitored for survival over time. FIG. 3C shows that i-Fect complexed siFvEJ reduces the level of viral replication in mouse brain when administered 6 h post challenge. Mice were injected with siRNAs 6 h after JEV challenge and brain homogenates obtained 3 d later were titrated on BHK21 cell monolayers. Log plaque-forming units per brain is shown. Each symbol represents an individual mouse. FIG. 3D shows that transfection of Neuro 2a cells with JetSI/DOPE complexed siFvEJ results in inhibition of JEV replication. Neuro 2a cells were treated with siFvEJ or siLuc as in a using JetSI/DOPE instead of i-Fect to complex the siRNAs. Overlay histogram denotations are indicated. FIG. 1E shows that siFvEJ complexed with JetSI/DOPE protects mice against fatal encephalitis. Mice (ten per group) were injected IC with four LD50 of JEV and were treated either with 3.2 nmoles siLuc complexed with JetSI/DOPE after 30 min or with JetSI/DOPE complexed with siFvEJ after 30 min, 6 h, or 18 h after infection and monitored for survival over time. FIG. 3F shows that shFvEJ fails to protect against WNV-induced encephalitis. Mice (five per group) were injected with 2×105 TU of RV-G pseudotyped shLuc or shFvEJ lentiviruses and challenged 30 min later with four LD50 of WNV and monitored for survival over time. FIG. 3G shows that siFvEW protects mice against lethal WNV-induced encephalitis. Mice (ten per group) were infected IC with four LD50 of WNV, and 30 min or 6 h later they were also injected with 3.2 nmoles of either control siLuc or siFvEW complexed with JetSI/DOPE, and monitored for survival over time.

FIGS. 4A-4B show that FvE^{JW} protects against encephalitis induced by either JEV or WNV. FIG. 4A shows siRNA-transfected Neuro 2a cells were challenged with 10 moi of JEV (left panel) or WNV (right panel) and examined 72 h after infection for viral replication by flow cytometry. Overlay histograms of uninfected Neuro 2a cells (grey filled histogram) and JEV- or WNV-infected Neuro 2a cells transfected prior to infection with JetSI/DOPE/siLuc (open histogram with dashed lines), LIPOFECTAMINE®/siFvE^{JW} (open histogram with thin solid line) or JetSI/DOPE/siFvE^{JW} (open histogram with thick solid line) are shown. FIG. 4B shows that FvEJW siRNA protects mice against both JEV and WNV-induced encephalitis. Mice were injected IC with four LD50 of JEV (left) or WNV (right), and after 30 min or 6 h they were also injected at the same spot with 3.2 nmoles of either siLuc or FvEJW complexed with JetSI/DOPE and monitored for survival over time. Ten and five mice per group were used to test the effect of siRNA 30 min and 6 h postinfection, respectively.

FIGS. 5A-5B show that FvE^{J} does not induce type I IFN responses. FIG. 5A shows mock or lentivirally transduced Vero cells were challenged with JEV at a moi of 10 and viral replication monitored by flow cytometry 72 h later after staining with a JEV-specific antibody. Percent infected cells is indicated. FIG. 5B shows cDNA prepared from Neuro 2a cells stably transduced with shLuc- or shFvE^{J}- (left panel), or shLuc- or shFvE^{J}-injected mouse brains obtained 24 h after injection (middle panel) and siLuc- or siFvE^{J}-treated mouse brain samples obtained 4 h later (right panel) was subjected to RT-PCR to measure the induction of IFN-response genes. The PCR products were quantified by NIH Image J (version 1.32j) software. Normalized values obtained for the test samples were divided by that obtained with untreated Neuro 2a cells or the brain sample from untreated mice to determine the fold induction in mRNA levels for each of the genes.

FIGS. 6A-6B show the conserved flaviviral genomic regions selected for targeting. FIG. 6A shows regions of the flaviviral genome selected for targeting. Targets include the capsid (or core), envelope, and NS3 genes, and 3′ untranslated regions of the flavivirus polyprotein mRNA. FIG. 6B shows the diagram of the lentivirus construct used to express the siRNAs of the present invention.

FIG. 7 shows FvE siRNA production in BHK21 cells. On the left, the flavivirus envelope gene (FvE) siRNA transfected cells were examined for GFP expression by flow cytometry. The percentage of stably transduced cells is shown in each panel. On the right modified Northern blot analysis shows FvE siRNA expression.

FIGS. 8A-8C show that inhibition of flaviviruses is most effectively accomplished through targeting of the flavivirus envelope gene. FIG. 8A shows inhibition of dengue virus replication by siRNAs. BHK21 cells were transfected with siRNAs and infected with virus after 24 hours. Virus replication was measured two days later by flow cytometry. The percentage of cells infected with virus is shown in each panel. The transfected siRNAs are (top, left to right) Ig control, mock transfected, GFP, flavivirus envelope (FvE); (bottom, left to right) Dengue E4 (D-E4), Dengue E3 (D-E3), flavivirus capsid (FvC) and flavivirus RNA polymerase (FvR1). FIG. 8B shows cross-species protection by different siRNAs. BHK-21 cells were mock transfected, transfected with luciferase, flavivirus envelope (FvE), flavivirus capsid (FvC) or flavivirus non-structural protein 3 (FvNS3). Cells were infected with Dengue virus, Japanese Encephalitis virus or West Nile virus at an moi of 1. Cells were analyzed by flow cytometry for GFP expression. The percentage of GFP positive cells is indicated in each panel. FIG. 8C shows duration of protection against flavivirus infection by siRNA at difference mois. Cells were transfected with a lentivirus shRNA expression vector expressing shRNAs for luciferase (Lenti-luci), flavivirus envelope gene (Lenti-FvE), flavivirus capsid gene (Lenti-FvC) or flavivirus NTPase (Lenti-NS3). Cells were infected with Dengue, Japanese Encephalitis or West Nile virus at varying mois. Percentage inhibition of the virus was measured at 48, 60 and 72 hours post infection.

FIG. 9 shows FvE shRNA protects mouse brains. Mice were treated with Luc-shRNA (left) and FvE-shRNA (right). The FvE shRNA-treated mice show an absence of virus in their brains.

### DETAILED DESCRIPTION OF THE INVENTION

The present invention is directed to methods of treating and/or preventing flavivirus infection. In one embodiment, the flavivirus infection is West Nile virus (WNV), Japanese Encephalitis virus (JEV), Dengue virus or St. Louis Encephalitis virus infection using double-stranded siRNAs designed to interfere with the expression of viral proteins.

Accordingly, the invention provides siRNAs, pharmaceutical compositions comprising said siRNAs, in vitro and in vivo methods of inhibiting expression of flavivirus, and methods of treating and/or preventing flavivirus infection.

The invention is based upon a finding that flavivirus infection was inhibited and prevented by the use of intracranial administration of siRNAs targeting flavivirus in an established animal model of flavivirus infection. We have previously shown that siRNA targeting the flavivirus capsid gene is effective in preventing Dengue virus replication in hamster cells [U.S. Provisional Application No. 60/488,501].

The family Flaviviridae includes the flaviviruses, hepatitis C virus (HCV), the animal pathogenic pestiviruses, and likely the GB virus A (GBV-A), GBV-B and GBV C/hepatitis G viruses (Murphy et al., Virus Taxonomy. Sixth Report of the International Committee on Taxonomy of Viruses, pp. 424-426, 1995; Vienna & New York: Springer-Verlag. A large number of flaviviruses are associated with human disease, and the epidemiology and pathology of three of these, West Nile virus (WNV), Dengue virus (DEN), and Japanese Encephalitis virus (JEV), are briefly summarized here.

West Nile virus is a mosquito borne pathogen associated with fever and encephalitis. WNV was first identified in Uganda in 1937 (Smithburn, et al., 1940. American Journal of Tropical Medicine, 20:471-92, 1940). Although outbreaks of WNV have been sporadic and associated with mild illness since its discovery, the frequency and severity of WNV disease, in horses as well as in humans, has increased since the mid 1990s (Petersen & Roehrig, Emerg Infect Dis. 7:611-4, 2001). Outbreaks have occurred in Romania (1996), Morocco (1996), Tunisia (1997), Italy (1998), Russia (1999), Israel (1999 and 2000) and the U.S. (1999, and each summer since). The outbreak in the state of New York in 1999 appears to mark the beginning of the spread of WNV throughout the U.S. In 1999, there were a total of 62 reported human cases isolated to the state of New York, 59 of which required hospitalization. In 2000, there were 21 cases in three states, increasing to 66 cases in ten states in 2001. The CDC, in 2002, reported 4156 laboratory-positive human cases over 38 states (anonymous 2003 on-line posting date, West Nile Virus, Centers for Disease Control). In 2003, there were 9862 human cases reported in 45 states, including 264 deaths (anonymous 2005 on-line posting date, West Nile Virus, Centers for Disease Control). Transmission involves cyclic transfer from mosquitoes of the genus Culex to birds and back. Humans and horses are dead-end hosts (Campbell, et al., Lances Infect Dis. 2:519-29, 2002).

Approximately 20% of individuals infected with WNV develop fever, as estimated by a serological survey conducted subsequent to the 1999 New York outbreak (Mostashari, et al., Lances, 358:261-4, 2001). This study estimates that the total number of infections during this period was 8,200 of which 62 were reported. The fever is sometimes accompanied by weakness, nausea, headache, myalgia, arthralgia, and rash. About 1 in 150 infections results in neurological disease such as encephalitis or meningitis (Mostashari, et al., Lances, 358:261-4, 2001; Petersen & Martin, Ann Intern Med. 137:173-9, 200). Of the 59 WNV patients hospitalized in New York in 1999, 54 were diagnosed with encephalitis or meningitis; 12% of these hospitalized patients later died. In 2002, 211 of the reported cases resulted in death (approximately a 6% fatality rate). The greatest risk factor for death is advanced age (Nash, et al., N Engl J Med. 344:1807-14, 2001). Significantly, there are currently no approved antiviral therapies for WNV; treatment is supportive.

Dengue virus infects approximately 100 million people a year. It is endemic in virtually all the tropic areas of the world. There are four serotypes of DEN (Dengue type 1-4). All are spread primarily by the mosquito Aedes aegypti, which lives in close proximity to humans (i.e. a “domestic” mosquito). Unlike the case for most flaviviruses, humans are a natural host for dengue, and can produce high enough-titers in the blood to continue the transmission cycle (Burke & Monath, 2001, supra; Gibbons & Vaughn, Bmj. 324.1563-6, 2002, and Solomon & Mallewa 2001. J Infect. 42:104-15, 2001).

DEN infection may result in one of several syndromes (McBride & Bielefeldt-Oluann, Microbes Infect. 2:1041-50, 2000). Dengue infection is characterized by fever, headache and rash. A more severe form, Dengue hemorrhagic fever (DHF) may include increased vascular permeability and leakage of plasma from blood vessels into tissue. Mild hemorrhage may also occur. DHF is graded on a scale of I through IV. Grade II includes greater bleeding (gum, nose, GI tract), while grades III and IV feature increased vascular leakage, accompanied by loss of blood pressure and shock. Grades III and IV are also known as Dengue shock syndrome. DHF is more likely to occur when DEN infection is followed by a second infection of a different serotype. This may be due to the presence of circulating antibody that reacts with, but does not neutralize, the second infecting strain. The presence of these antibodies allows antibody dependent enhancement of infection of macrophages, which take up antibody-bound DEN via their Fc receptors. It is postulated that macrophage infection results in increased T-cell activation and cytokine production, leading to severe immunopathology (Halstead, S. B., science 239:476-81, 1988). This model does not explain, however, the relative rarity of DHF even in patients experiencing a second DEN infection, or the occasional appearance of DHF during primary DEN infection. Other theories of DHF pathogenesis include the possibility of virulence factors present only in specific DEN strains or “quasispecies,” or the possibility of an autoimmune response elicited by the similarity of DEN antigens to various human clotting factors (Bielefeldt-Ohmann, H., Trends Microbiol. 5:409-13, 1997, Leitmeyer, et al., J Virol. 73.4738-4, 1999, and Markoff, et al., J Infect Dis. 164:294-30, 1991).

Japanese Encephalitis virus is endemic in much of Southeast Asia, ranging from Japan and Korea at its northern range, to India in the west, and Indochina and Indonesia to the South. Sporadic cases have also been reported as far south as Papua New Guinea and Australia. Annually, there are approximately 35,000 cases and 10,000 deaths, and these figures may underestimate the true toll of the disease due to incomplete surveillance and reporting. JEV is a member of an antigenic complex and clade that also include WNV. It is spread primarily by the mosquito Culex tritaeniorhynchus, cycling through its natural viremic hosts, pigs and birds.

The flaviviruses are small enveloped viruses that contain a single, positive-sense RNA genome of approximately 11 kilobases (kb). The RNA is capped at its 5′ end, but not 3′ polyadenylated. The RNA encodes a single large open reading frame (ORF) that is processed into 10 subunits that comprise the structural components of the virion and the viral replication complex (Lindenbach & Rice, 2001, Flaviviridae: The Viruses and Their Replication, p. 991-1041; in D. M. Knipe, and P. M. Howley (eds), c, Fourth ed. Lippincott Williams & Wilkins, Philadelphia). The flaviviruses all possess a common organization to the coding sequence of the genome. The structural subunits are located at the 5′ end. These include the core or capsid (C), membrane (prM/M), and envelope (E) proteins. These are followed by the non-structural proteins NS1, NS2A, NS2B, NS3, NS4A, NS4B, and NS5.

NS2B and NS3 function as the serine protease that is responsible for processing much of the viral polyprotein. NS5, the most highly conserved of the flavivirus proteins, acts as the RNA dependent RNA polymerase necessary for viral replication, and may also function as a methyltransferase that provides the genomic 5′ cap. The other members of the non-structural group are largely hydrophobic and of unknown function (Id).

Flavivirus infection of the host cell begins via attachment of the E-protein to a cellular receptor. Definitive identification of a receptor for any of the flavivirus species is still absent, but glycosaminoglycans appear to be involved in the initial attachment (Chen, et al., Nat Med. 3:866-71, 1997). Entry of the virus into the host cell probably occurs by receptor mediated endocytosis, followed by low-pH dependent fusion of the virion with the endosome membrane, releasing the nucleocapsid and genomic RNA into the cytoplasm (Lindenbach & Rice, 2001, supra; Kuhn, et al., Cell. 108:717-2, 2002).

Translation of the RNA by the host cell follows, and the polyprotein is cleaved into its constituent subunits by a combination off host cell ER resident protease and the NS2B/NS3 virally encoded serine protease. Replication of the genomic RNA occurs through a negative sense intermediate, and can be detected as early as three hours after infection in the case of YFV. Flavivirus infection induces a proliferation of ER membranes in the host cell and the formation of “smooth membrane structures,” that are groups of vesicle-like structures in the ER lumen. The smooth membrane structures co-localize with double-stranded RNA (presumably the replicative intermediate), as well as NS1 and NS3, and are believed to be the sites of RNA replication. NS2B and NS3, the constituents of the viral protease, localize to an adjacent region of induced membranes (dubbed “convoluted membranes”), suggesting that polyprotein processing and nucleic acid replication are spatially separated within the infected cell (Westaway, et al., J Virol. 71:6650-61, 1997).

Assembly and release of virions largely remains a black box. Cis-acting packaging signals in the RNA have not been identified, although the viral nucleocapsid protein C has been shown to interact with the 5′ and 3′ ends of the genome (Khromykh & Westaway, Arch Virol. 141:685-99, 1996). The envelope is most likely acquired by budding of the nucleocapsid precursor into the ER. At a later point in virus maturation, the prM protein is cleaved into the mature form (M) by the cellular protease furin (Stadler, et al., J Virol. 71:8475-81, 1997). It is currently believed that prM functions to prevent the E protein from undergoing the low pH dependent conformational change while in the cell. In agreement with this hypothesis, prevention of prM cleavage results in the release of virus particles that are less infectious than wild-type (Heinz & Allison, Adv Virus Res. 55:231-69, 2000).

Infection of the host is thought to begin in the Langerhans cells of the skin following the bite of a carrier arthropod. Viral replication continues in the regional tissue and lymph nodes, which results in the dissemination of the virus into the bloodstream. Replication then proceeds at several sites, including connective tissue, smooth muscle, liver and spleen. Neural invasion appears to occur through the olfactory epithelium in experimentally infected rodents. It is unclear if this is the primary route used by the virus to gain access to the CNS in infected humans (McMinn et al., Virology. 220:414-23, 1996; Monath, et al., Lab Invest. 48:399-410, 1983).

The present invention provides siRNAs, pharmaceutical compositions comprising said siRNAs, in vitro and in vivo methods of inhibiting expression of flavivirus and methods of treating and/or preventing flavivirus infection. Accordingly, the invention provides an isolated siRNA comprising a sense RNA strand and an antisense RNA strand, or a single RNA strand, wherein the sense and the antisense RNA strands, or the single RNA strand, form an RNA duplex, and wherein the RNA strand comprises a nucleotide sequence identical to a target sequence of about 19 to about 25 contiguous nucleotides in flavivirus mRNA or mutant or variant thereof. The flavivirus mRNA useful according to the invention refers to any known nucleic acid that is part of a flavivirus genome. For example, sequences identified as GenBank ID Nos. NC_{—}001563 (WNV); NC_{—}001474 (Dengue); and NC_{—}001437 (JEV). In one embodiment, the siRNA target sequences are directed to the genes encoding the capsid protein (also called the core protein), C; the envelope protein, E; the non-structural protein 3, NS3, untranslated regions or any combination thereof. In one embodiment, the siRNA target sequences do not include the gene encoding the capsid protein. In another embodiment, the siRNA target sequences include the gene encoding the capsid protein in combination with the envelope protein, the non-structural protein 3, and/or untranslated regions. In yet another embodiment, the siRNAs are as in Table 1. In one embodiment, the siRNAs comprise SEQ ID NOS: 11-16 from Table 1 or any combination thereof. Alternatively, the siRNAs comprise SEQ ID NOS: 1-95 from Table 1 or any combination thereof.

“Short interfering RNA” (siRNA), also referred to herein as “small interfering RNA” is defined as an agent which functions to inhibit expression of a target gene, e.g., by RNAi. An siRNA may be chemically synthesized, it may be produced by in vitro transcription, or it may be produced within a host cell.

In one embodiment, siRNA is a double stranded RNA (dsRNA) molecule of about 15, 16, 17, 18, 19, 20, 21, 22, 23, 24, 25, 26, 27, 28, or 30 nucleotides in length. In one embodiment the length is about 15 to about 28 nucleotides. In another embodiment, the length is about 19, 20, 21, 22, 23, 24, or 25 nucleotides in length. In yet another embodiment the length is about 19, 20, 21, 22, or 23 nucleotides in length, and may contain a 3′ and/or 5′ overhang on each strand having a length of about 1, 2, 3, 4, or 5 nucleotides. The length of the overhang is independent between the two strands, i.e., the length of the over hang on one strand is not dependent on the length of the overhang on the second strand. In one embodiment, the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

TABLE 1

| | | | | | |
|---|---|---|---|---|---|
| Target | Seq | Sense | Seq | Antisense | Seq |
| Sequence | ID | Strand | ID | strand | ID |

| | | | | | |
|---|---|---|---|---|---|
| CTATCAATATGCT | 96 | CUAUCAAUAUGCU | 1 | CGCGUUCAGCAUA | 2 |
| GAACGCG | | GAACGCG | | UUGAUAG | |
| CGGATGTGGACTT | 97 | CGGAUGUGGACUU | 3 | CCCGAAAAGUCCA | 4 |
| TTCGGG | | UUCGGG | | CAUCCG | |
| GACAGAAGGTGGT | 98 | GACAGAAGGUGGU | 5 | AUCAAACACCACC | 6 |
| GTTTGAT | | GUUUGAU | | UUCUGUC | |
| CAGCATATTGACA | 99 | CAGCAUAUUGACA | 7 | CCCAGGUGUCAAU | 8 |
| CCTGGG | | CCUGGG | | AUGCUG | |
| GGACTAGAGGTTA | 100 | GGACUAGAGGUUA | 9 | CUCCUCUAACCUC | 10 |
| GAGGAG | | GAGGAG | | UAGUCC | |
| GGATGTGGACTTT | 101 | GGAUGUGGACUUU | 11 | UCCCGAAAAGUCC | 12 |
| TCGGGA | | UCGGGA | | ACAUCC | |
| GGCTGCGGACTGT | 102 | GGCUGCGGACUGU | 13 | UUCCAAACAGUCC | 14 |
| TTGGAA | | UUGGAA | | GCAGCC | |
| GGGAGCATTGACA | 103 | GGGAGCAUUGACA | 15 | UGCACAUGUGUCA | 16 |
| CATGTGCA | | CAUGUGCA | | AUGCUCCC | |
| ACACAACATGGAA | 104 | ACACAACAUGGAA | 32 | CUAUUGUUCCAUG | 33 |
| CAATAG | | CAAUAG | | UUGUGU | |
| CATAGAAGCAGAA | 105 | CAUAGAAGCAGAA | 34 | UGGAGGUUCUGCU | 35 |
| CCTCCA | | CCUCCA | | UCUAUG | |
| GGAACATCAGGCT | 106 | GGAACAUCAGGCU | 36 | UAUUGGUGAGCCU | 37 |
| CACCAATA | | CACCAAUA | | GAUGUUCC | |
| GGGCTTTATGGCA | 107 | GGGCUUUAUGGCA | 38 | UGACUCCAUUGCC | 39 |
| ATGGAGTCA | | AUGGAGUCA | | AUAAAGCCC | |
| TCTGCCACAGATC | 108 | UCUGCCACAGAUC | 40 | UCUUUGAUGAUCU | 41 |
| ATCAAAGA | | AUCAAAGA | | GUGGCAGA | |
| GTGGCTGCTGAGA | 109 | GUGGCUGCUGAGA | 42 | UUCAGCCAUCUCA | 43 |
| TGGCTGAA | | UGGCUGAA | | GCAGCCAC | |
| CTCACCCACAGGC | 110 | CUCACCCACAGGC | 44 | AGACAUCAGCCUG | 45 |
| TGATGTCT | | UGAUGUCU | | UGGGUGAG | |
| GTGATGGATGAGG | 111 | GUGAUGGAUGAGG | 46 | GAAAUGAGCCUCA | 47 |
| CTCATTTC | | CUCAUUUC | | UCCAUCAC | |
| GATACGAATGGAT | 112 | GAUACGAAUGGAU | 48 | AUUCUGUGAUCCA | 49 |
| CACAGAAT | | CACAGAAU | | UUCGUAUC | |
| GGAAGTCAGAGGG | 113 | GGAAGUCAGAGGG | 50 | UUUGUGUACCCUC | 51 |
| TACACAAA | | UACACAAA | | UGACUUCC | |
| GGTCACCATGAAG | 114 | GGUCACCAUGAAG | 52 | ACUCCACUCUUCA | 53 |
| AGTGGAGT | | AGUGGAGU | | UGGUGACC | |
| ACTCCACGCACGA | 115 | ACUCCACGCACGA | 54 | AACACAUCUCGUG | 55 |
| GATGTGTT | | GAUGUGUU | | CGUGGAGU | |
| CCATGGCCATGAC | 116 | CCAUGGCCAUGAC | 56 | UAGUGUCAGUCAU | 57 |
| TGACACTA | | UGACACUA | | GGCCAUGG | |
| GCCATTTGGTTCAT | 117 | GCCAUUUGGUUCA | 58 | AAGCCACAUGAAC | 59 |
| GTGGCTT | | UGUGGCUU | | CAAAUGGC | |
| TGGACCTGGCTGT | 118 | UGGACCUGGCUGU | 60 | AUUCUCAAACAGC | 61 |
| TTGAGAAT | | UUGAGAAU | | CAGGUCCA | |
| AATATCAAACACC | 119 | AAUAUCAAACACC | 62 | UCGGUGGUGGUGU | 63 |
| ACCACCGA | | ACCACCGA | | UUGAUAUU | |
| AAAGCTTTGAAAC | 120 | AAAGCUUUGAAAC | 64 | CCAGCUUAGUUUC | 65 |
| TAAGCTGG | | UAAGCUGG | | AAAGCUUU | |
| AAGAAGGGCCTCT | 121 | AAGAAGGGCCUCU | 66 | UCUCUGGUAGAGG | 67 |
| ACCAGAGA | | ACCAGAGA | | CCCUUCUU | |
| AAGGGATTATCCC | 122 | AAGGGAUUAUCCC | 68 | AGAGGGCUGGGAU | 69 |
| AGCCCTCT | | AGCCCUCU | | AAUCCCUU | |
| AAGAGGTGGCTGG | 123 | AAGAGGUGGCUGG | 70 | UAAUAUGACCAGC | 71 |
| TCATATTA | | UCAUAUUA | | CACCUCUU | |
| AAATGAAGAGCAG | 124 | AAAUGAAGAGCAG | 72 | CUUUUGUCCUGCU | 73 |
| GACAAAAG | | GACAAAAG | | CUUCAUUU | |
| AAATTGGATACAG | 125 | AAAUUGGAUACAG | 74 | GUCUCUUUCUGUA | 75 |
| AAAGAGAC | | AAAGAGAC | | UCCAAUUU | |
| AAACACAACATGG | 126 | AAACACAACAUGG | 76 | CUAUUGUUCCAUG | 77 |
| AACAATAG | | AACAAUAG | | UUGUGUUU | |
| AACATAGAAGCAG | 127 | AACAUAGAAGCAG | 78 | UGGAGGUUCUGCU | 79 |
| AACCTCCA | | AACCUCCA | | UCUAUGUU | |
| AAAGGGAAGACTG | 128 | AAAGGGAAGACUG | 80 | GAACCAAACAGUC | 81 |
| TTTGGTTC | | UUUGGUUC | | UUCCCUUU | |
| AAAAGGAAAAGTT | 129 | AAAAGGAAAAGUU | 82 | AGACCCACAACUU | 83 |
| GTGGGTCT | | GUGGGUCU | | UUCCUUUU | |
| AATGGCCATCAGT | 130 | AAUGGCCAUCAGU | 84 | UCAUCUCCACUGA | 85 |
| GGAGATGA | | GGAGAUGA | | UGGCCAUU | |
| AAAGGTGAGAAGC | 131 | AAAGGUGAGAAGC | 86 | GCUGCAUUGCUUC | 87 |
| AATGCAGC | | AAUGCAGC | | UCACCUUU | |
| AAAAGCAAGAAGT | 132 | AAAAGCAAGAAGU | 88 | GGACAACUACUUC | 89 |
| AGTTGTCC | | AGUUGUCC | | UUGCUUUU | |
| AAAATTGGAATAG | 133 | AAAAUUGGAAUAG | 90 | GAGGACACCUAUU | 91 |
| GTGTCCTC | | GUGUCCUC | | CCAAUUUU | |
| AAAATCCTTACAA | 134 | AAAAUCCUUACAA | 92 | CCCACGUUUUGUA | 93 |
| AACGTGGG | | AACGUGGG | | AGGAUUUU | |
| AAATCCTTACAAA | 135 | AAAUCCUUACAAA | 94 | GCCCACGUUUUGU | 95 |
| ACGTGGGC | | ACGUGGGC | | AAGGAUUU | |

The invention also provides a recombinant vector comprising nucleic acid sequences for expressing an siRNA comprising a sense RNA strand and an antisense RNA strand, or a single strand, wherein the sense and the antisense RNA strands, or the single strand, form an RNA duplex, and wherein the RNA strand comprises a nucleotide sequence identical to a target sequence of about 19 to about 25 contiguous nucleotides in flavivirus mRNA or mutant or variant thereof.

The invention also provides a pharmaceutical composition comprising at least one siRNA and a pharmaceutically acceptable carrier, wherein the siRNA comprises a sense RNA strand and an antisense RNA strand or a single strand, wherein the sense and the antisense RNA strands or the single strand form an RNA duplex, and wherein the RNA strand comprises a nucleotide sequence identical to a target sequence of about 19 to about 25 contiguous nucleotides in flavivirus mRNA, or an alternative splice form, mutant or cognate thereof.

Delivery of siRNA Agents

Methods of delivering siRNA of the present invention, or vectors containing siRNA of the present invention, to the target cells, such as neuronal cells, macrophages and all other body cells, include injection of a composition containing the siRNA, or directly contacting the target cell, with a composition comprising an siRNA. In another embodiment, an siRNA may be injected directly into any blood vessel, such as vein, artery, venule or arteriole, via methods including but not limited to hydrodynamic injection or catheterization. Administration may be by a single injection or by two or more injections. The siRNA is delivered in a pharmaceutically acceptable carrier. One or more siRNAs targeting flavivirus may be used simultaneously.

In one embodiment, only one siRNA that targets flavivirus is used. The delivery or administration of the siRNA is in one embodiment performed in free form, i.e. without the use of vectors. In another embodiment, a mixture of siRNAs targeting either the same viral gene or at least 2, 3, 4, 5 or up to at least 10 different flavivirus genes or gene variants are used.

In one embodiment, the compositions of the invention are provided as a surface component of a lipid aggregate, such as a liposome, or are encapsulated by a liposome. Liposomes, which can be unilamellar or multilamellar, can introduce encapsulated material into a cell by different mechanisms. For example, the liposome can directly introduce its encapsulated material into the cell cytoplasm by fusing with the cell membrane. Alternatively, the liposome can be compartmentalized into an acidic vacuole (i.e., an endosome) and its contents released from the liposome and out of the acidic vacuole into the cellular cytoplasm. In one embodiment the invention features a lipid aggregate formulation of the compounds described herein, including phosphatidylcholine (of varying chain length; e.g., egg yolk phosphatidylcholine), cholesterol, a cationic lipid, and 1,2-distearoyl-sn-glycero3-phosphoethanolamine-polyethyleneglycol-2000 (DSPE-PEG2000). The cationic lipid component of this lipid aggregate can be any cat ionic lipid known in the art such as dioleoyl 1,2-diacyl trimethylammonium-propane (DOTAP). In another embodiment, polyethylene glycol (PEG) is covalently attached to the compositions of the present invention. The attached PEG can be any molecular weight but is typically between 2000-50,000 daltons. In one embodiment for targeting macrophages for delivery of siRNA, liposomes containing of phosphatidyl serine may be used since macrophage engulfment via the phosphatidyl serine receptor promotes an anti-inflammatory response by increasing TGF-beta1 secretion (Huynh, M. L. et al. (2002) J. Cell Biol. 155, 649). Therefore, when the macrophages are successfully transfected, not only will the target genes be silenced, but the macrophage will also be induced to secrete anti-inflammatory cytokines.

In another embodiment, for delivery to a macrophage, a polyG tail, e.g., a 5-10 nucleotide tail, may be added to the 5′ end of the sense strand of the siRNA, which will enhance uptake via the macrophage scavenger receptor.

In another embodiment of the invention, the siRNA of the invention may be transported or conducted across biological membranes using carrier polymers which comprise, for example, contiguous, basic subunits, at a rate higher than the rate of transport of siRNA molecules which are not associated with carrier polymers. Combining a carrier polymer with siRNA, with or without a cationic transfection agent, results in the association of the carrier polymer and the siRNA. The carrier polymer may efficiently deliver the siRNA, across biological membranes both in vitro and in vivo. Accordingly, the invention provides methods for delivery of an siRNA, across a biological membrane, e.g., a cellular membrane including, for example, a nuclear membrane, using a carrier polymer. The invention also provides compositions comprising an siRNA in association with a carrier polymer.

The term “association” or “interaction” as used herein in reference to the association or interaction of an siRNA and a carrier polymer, refers to any association or interaction between an siRNA with a carrier polymer, e.g., a peptide carrier, either by a direct linkage or an indirect linkage. An indirect linkage includes an association between an siRNA and a carrier polymer wherein said siRNA and said carrier polymer are attached via a linker moiety, e.g., they are not directly linked. Linker moieties include, but are not limited to, e.g., nucleic acid linker molecules, e.g., biodegradable nucleic acid linker molecules. A nucleic acid linker molecule may be, for example, a dimer, trimer, tetramer, or longer nucleic acid molecule, for example an oligonucleotide of about 2, 3, 4, 5, 6, 7, 8, 9, 10, 11, 12, 13, 14, 15, 16, 17, 18, 19, 20, 21, 22, 23, 24, 25, or more nucleotides in length.

A direct linkage includes any linkage wherein a linker moiety is not required. In one embodiment, a direct linkage includes a chemical or a physical interaction wherein the two moieties, the siRNA and the carrier polymer, interact such that they are attracted to each other. Examples of direct interactions include non-covalent interactions, hydrophobic/hydrophilic, ionic (e.g., electrostatic, coulombic attraction, ion-dipole, charge-transfer), Van der Waals, or hydrogen bonding, and chemical bonding, including the formation of a covalent bond. Accordingly, in one embodiment, the siRNA and the carrier polymer are not linked via a linker, e.g., they are directly linked. In a further embodiment, the siRNA and the carrier polymer are electrostatically associated with each other.

The term “polymer” as used herein, refers to a linear chain of two or more identical or non-identical subunits joined by covalent bonds. A peptide is an example of a polymer that can be composed of identical or non-identical amino acid subunits that are joined by peptide linkages.

The term “peptide” as used herein, refers to a compound made up of a single chain of D- or L-amino acids or a mixture of D- and L-amino acids joined by peptide bonds. Generally, peptides contain at least two amino acid residues and are less than about 50 amino acids in length.

The term “protein” as used herein, refers to a compound that is composed of linearly arranged amino acids linked by peptide bonds, but in contrast to peptides, has a well-defined conformation. Proteins, as opposed to peptides, generally consist of chains of 50 or more amino acids.

“Polypeptide” as used herein, refers to a polymer of at least two amino acid residues and which contains one or more peptide bonds. “Polypeptide” encompasses peptides and proteins, regardless of whether the polypeptide has a well-defined conformation.

In one embodiment, carrier polymers in accordance with the present invention contain short-length polymers of from about 6 to up to about 25 subunits. The carrier is effective to enhance the transport rate of the siRNA across the biological membrane relative to the transport rate of the biological agent alone. Although exemplified polymer compositions are peptides, the polymers may contain non-peptide backbones and/or subunits as discussed further below.

In an important aspect of the invention, the carrier polymers of the invention are particularly useful for transporting biologically active agents across cell or organelle membranes, when the siRNAs are of the type that require trans-membrane transport to exert their biological effects. Typically, the carrier polymer used in the methods of the invention includes a linear backbone of subunits. The backbone will usually comprise heteroatoms selected from carbon, nitrogen, oxygen, sulfur, and phosphorus, with the majority of backbone chain atoms usually consisting of carbon. Each subunit may contain a sidechain moiety that includes a terminal guanidino or amidino group.

Although the spacing between adjacent sidechain moieties will usually be consistent from subunit to subunit, the polymers used in the invention can also include variable spacing between sidechain moieties along the backbone.

The sidechain moieties extend away from the backbone such that the central guanidino or amidino carbon atom (to which the NH₂ groups are attached) is linked to the backbone by a sidechain linker that typically contains at least 2 linker chain atoms, in one embodiment, the linker contain 2 to 5 chain atoms, such that the central carbon atom is the third to sixth chain atom away from the backbone. The chain atoms are usually provided as methylene carbon atoms, although one or more other atoms such as oxygen, sulfur, or nitrogen can also be present. In one embodiment, the sidechain linker between the backbone and the central carbon atom of the guanidino or amidino group is 4 chain atoms long, as exemplified by an arginine side chain.

The carrier polymer sequence of the invention can be flanked by one or more non-guanidino/non-amidino subunits, or a linker such as an aminocaproic acid group, which do not significantly affect the rate of membrane transport of the corresponding polymer-containing conjugate, such as glycine, alanine, and cysteine, for example. Also, any free amino terminal group can be capped with a blocking group, such as an acetyl or benzyl group, to prevent ubiquitination in vivo.

The carrier polymer of the invention can be prepared by straightforward synthetic schemes. Furthermore, the carrier polymers are usually substantially homogeneous in length and composition, so that they provide greater consistency and reproducibility in their effects than heterogenous mixtures.

According to an important aspect of the present invention, association of a single carrier polymer to an siRNA is sufficient to substantially enhance the rate of uptake of an siRNA across biological membranes, even without requiring the presence of a large hydrophobic moiety in the conjugate. In fact, attaching a large hydrophobic moiety may significantly impede or prevent cross-membrane transport due to adhesion of the hydrophobic moiety to the lipid bilayer. Accordingly, the present invention includes carrier polymers that do not contain large hydrophobic moieties, such as lipid and fatty acid molecules.

In one embodiment, the transport polymer is composed of D- or L-amino acid residues. Use of naturally occurring L-amino acid residues in the transport polymers has the advantage that break-down products should be relatively non-toxic to the cell or organism. Typical amino acid subunits are arginine (alpha-amino-delta-guanidinovaleric acid) and alpha-amino-epsilon-amidinohexanoic acid (isosteric amidino analog). The guanidinium group in arginine has a pKa of about 12.5.

More generally, each polymer subunit can contain a highly basic sidechain moiety which (i) has a pKa of greater than 11, and in one embodiment, 12.5 or greater, and (ii) contains, in its protonated state, at least two geminal amino groups (NH₂) which share a resonance-stabilized positive charge, which gives the moiety a bidentate character.

Other amino acids, such as alpha-amino-beta-guanidinopropionic acid, alpha-amino-gamma-guanidinobutyric acid, or alpha-amino-epsilon-guanidinocaproic acid can also be used (containing 2, 3 or 5 linker atoms, respectively, between the backbone chain and the central guanidinium carbon).

D-amino acids may also be used in the transport polymers. Compositions containing exclusively D-amino acids have the advantage of decreased enzymatic degradation. However, they may also remain largely intact within the target cell. Such stability is generally not problematic if the agent is biologically active when the polymer is still attached. For agents that are inactive in conjugate form, a linker that is cleavable at the site of action (e.g., by enzyme- or solvent-mediated cleavage within a cell) should be included to promote release of the agent in cells or organelles.

Any peptide, e.g., basic peptide, or fragment thereof, which is capable of crossing a biological membrane, either in vivo or in vitro, is included in the invention. These peptides can be synthesized by methods known to one of skill in the art. For example, several peptides have been identified which may be used as carrier peptides in the methods of the invention for transporting siRNAs across biological membranes. These peptides include, for example, the homeodomain of antennapedia, a Drosophila transcription factor (Wang et al., (1995) PNAS USA., 92, 3318-3322); a fragment representing the hydrophobic region of the signal sequence of Kaposi fibroblast growth factor with or without NLS domain (Antopolsky et al. (1999) Bioconj. Chem., 10, 598-606); a signal peptide sequence of Caiman crocodylus Ig(5) light chain (Chaloin et al. (1997) Biochem. Biophys. Res. Comm., 243, 601-608); a fusion sequence of HIV envelope glycoprotein gp4114, (Morris et al. (1997) Nucleic Acids Res., 25, 2730-2736); a transportan A-achimeric 27-mer consisting of N-terminal fragment of neuropeptide galanine and membrane interacting wasp venom peptide mastoporan (Lindgren et al., (2000), Bioconjugate Chem., 11, 619-626); a peptide derived from influenza virus hemagglutinin envelop glycoprotein (Bongartz et al., 1994, Nucleic Acids Res., 22, 468 1 4688); RGD peptide; and a peptide derived from the human immunodeficiency virus type-1 (“HIV-1”). Purified HIV-1 TAT protein is taken up from the surrounding medium by human cells growing in culture (A. D. Frankel and C. O. Pabo, (1988) Cell, 55, pp. 1189-93). TAT protein trans-activates certain HIV genes and is essential for viral replication. The full-length HIV-1 TAT protein has 86 amino acid residues. The HIV tat gene has two exons. TAT amino acids 1-72 are encoded by exon 1, and amino acids 73-86 are encoded by exon 2. The full-length TAT protein is characterized by a basic region which contains two lysines and six arginines (amino acids 47-57) and a cysteine-rich region which contains seven cysteine residues (amino acids 22-37). The basic region (i.e., amino acids 47-57) is thought to be important for nuclear localization. Ruben, S. et al., J. Virol. 63: 1-8 (1989); Hauber, J. et al., J. Virol. 63 1181-1187 (1989); Rudolph et al. (2003) 278(13):11411. The cysteine-rich region mediates the formation of metal-linked dimers in vitro (Frankel, A. D. et al., Science 240: 70-73-(1988); Frankel, A. D. et al., Proc. Natl. Acad. Sci USA 85: 6297-6300 (1988)) and is essential for its activity as a transactivator (Garcia, J. A. et al., EMBO J. 7:3143 (1988); Sadaie, M. R. et al., J. Virol. 63: 1 (1989)). As in other regulatory proteins, the N-terminal region may be involved in protection against intracellular proteases (Bachmair, A. et al., Cell 56: 1019-1032 (1989).

In one embodiment of the invention, the basic peptide comprises amino acids 47-57 of the HIV-1 TAT peptide. In another embodiment, the basic peptide comprises amino acids 48-60 of the HIV-1 TAT peptide. In still another embodiment, the basic peptide comprises amino acids 49-57 of the HIV-1 TAT peptide. In yet another embodiment, the basic peptide comprises amino acids 49-57, 48-60, or 47-57 of the HIV-1-TAT peptide, does not comprise amino acids 22-36 of the HIV-1 TAT peptide, and does not comprise amino acids 73-86 of the HIV-1 TAT peptide. In still another embodiment, the specific peptides set forth in Table 2, below, or fragments thereof, may be used as carrier peptides in the methods and compositions of the invention.

TABLE 2

| | | SEQ |
|---|---|---|
| Peptide | Sequence | ID NO: |
| HIV-1 TAT (49-57) | RKKRRQRRR | 17 |
| HIV-1 TAT (48-60) | grkkrrqrrrtpq | 18 |
| HIV-1 TAT (47-57) | ygrkkrrqrrR | 19 |
| Kaposi fibroblast | AAV ALL PAV LLA LLA P +/− | 20 (NLS |
| growth factor | nuclear localization signal, | disclosed |
| | such as VQR KRQ KLMP | as SEQ ID |
| | | NO: 136) |
| of caiman crocodylus | MGL GLH LLV LAA ALQ GA | 21 |
| Ig(5) light chain | | |
| HIV envelope | GAL FLG FLG AAG STM GA +/− | 22 (NLS |
| glycoprotein gp41 | nuclear localization signal, such as | disclosed |
| | SPKKKRKVEAS (NLS of the SV40) | as SEQ ID |
| | | NO: 137) |
| Drosophila | RQI KIW FQN RRM KWK K amide | 23 |
| Antennapedia | | |
| RGD peptide | X-RGD-X | 24 |
| Influenza virus | glfeaiagfiengwegmidgggyc | 25 |
| hemagglutinin | | |
| envelop glycoprotein | | |
| transportan A | GWT LNS AGY LLG KIN LKA LAA | 26 |
| | LAK KIL | |
| Pre-S-peptide | (S)DH QLN PAF | 27 |
| Somatostatin | (S)FC YWK TCT | 28 |
| (tyr-3-octreotate) | | |

(s) Optional Serine for Coupling

italic=optional D isomer for stability

In another embodiment, the delivery is performed using an siRNA delivery system described in U.S. provisional patent application No. 60/601,950 filed Aug. 16, 2004, and U.S. Patent Application Publication No. 20040023902, incorporated herein by reference in their entirety. The method of targeted delivery both in vitro and in vivo of siRNAs into desired cells thus avoiding entry of the siRNA into other than intended target cells. The method allows treatment of specific cells with siRNAs limiting potential side effects of RNA interference caused by non-specific targeting of RNA interference. The method uses a complex or a fusion molecule comprising a cell targeting moiety and an siRNA binding moiety that is used to deliver the siRNA effectively into cells. For example, an antibody-protamine fusion protein when mixed with siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety may be either in the carboxyl-terminal or amino-terminal end of the construct or in the middle of the fusion protein.

In yet another embodiment, an active thiol at the 5′ end of the sense strand may be coupled to a cysteine reside added to the C terminal end of a basic peptide for delivery into the cytosol (such as a fragment of tat or a fragment of the Drosophila Antennapedia peptide). Internalization via these peptides bypasses the endocytic pathway and therefore removes the danger of rapid degradation in the harsh lysosomal environment, and may reduce the concentration required for biological efficiency compared to free oligonucleotides.

Other arginine rich basic peptides are also included for use in the present invention. For example, a TAT analog comprising D-amino acid- and arginine-substituted TAT(47-60), RNA-binding peptides derived from virus proteins such as HIV-1 Rev, and flock house virus coat proteins, and the DNA binding sequences of leucine zipper proteins, such as cancer-related proteins c-Fos and c-Jun and the yeast transcription factor GCN4, all of which contain several arginine residues (see Futaki, et al. (2001) J. Biol Chem 276(8):5836-5840 and Futaki, S. (2002) Int J. Pharm 245(1-2):1-7, which are incorporated herein by reference). In one embodiment, the arginine rich peptide contains about 4 to about 11 arginine residues. In another embodiment, the arginine residues are contiguous residues.

Subunits other than amino acids may also be selected for use in forming transport polymers. Such subunits may include, but are not limited to hydroxy amino acids, N-methyl-amino acids amino aldehydes, and the like, which result in polymers with reduced peptide bonds. Other subunit types can be used, depending on the nature of the selected backbone.

A variety of backbone types can be used to order and position the sidechain guanidino and/or amidino moieties, such as alkyl backbone moieties joined by thioethers or sulfonyl groups, hydroxy acid esters (equivalent to replacing amide linkages with ester linkages), replacing the alpha carbon with nitrogen to form an aza analog, alkyl backbone moieties joined by carbamate groups, polyethyleneimines (PEIs), and amino aldehydes, which result in polymers composed of secondary amines.

A more detailed backbone list includes N-substituted amide (CONR replaces CONH linkages), esters (CO₂), ketomethylene (COCH₂) reduced or methyleneamino (CH₂NH), thioamide (CSNH), phosphinate (PO₂RCH₂), phosphonamidate and phosphonamidate ester (PO₂RNH), retropeptide (NHCO), transalkene (CR.dbd.CH), fluoroalkene (CF.dbd.CH), dimethylene (CH₂2CH₂), thioether (CH₂S), hydroxyethylene (CH(OH)CH₂), methyleneoxy (CH₂O), tetrazole (CN₂4), retrothioamide (NHCS), retroreduced (NHCH₂), sulfonamido (SO₂NH), methylenesulfonamido, (CHRSO₂NH), retrosulfonamide (NHSO₂), and peptoids (N-substituted glycines), and backbones with malonate and/or gem-diaminoalkyl subunits, for example, as reviewed by Fletcher et al. (1998) and detailed by references cited therein. Peptoid backbones (N-substituted glycines) can also be used. Many of the foregoing substitutions result in approximately isosteric polymer backbones relative to backbones formed from α-amino acids.

Polymers are constructed by any method known in the art. Exemplary peptide polymers can be produced synthetically, for example, using a peptide synthesizer, such as Applied Biosystems Model 433, or the like, or can be synthesized recombinantly by methods well known in the art.

N-methyl and hydroxy-amino acids can be substituted for conventional amino acids in solid phase peptide synthesis. However, production of polymers with reduced peptide bonds requires synthesis of the dimer of amino acids containing the reduced peptide bond. Such dimers are incorporated into polymers using standard solid phase synthesis procedures. Other synthesis procedures are well known in the art.

In one embodiment of the invention, an siRNA and the carrier polymer are combined together prior to contacting a biological membrane. Combining the siRNA and the carrier polymer results in an association of the agent and the carrier. In one embodiment, the siRNA and the carrier polymer are not indirectly linked together. Therefore, linkers are not required for the formation of the duplex. In another embodiment, the siRNA and the carrier polymer are bound together via electrostatic bonding.

It is known that depending upon the expression vector and transfection technique used, only a small fraction of cells may effectively uptake the siRNA molecule. In order to identify and select these cells, antibodies against a cellular target can be used to determine transfection efficiency through immunofluorescence. Typical cellular targets include those which are present in the host cell type and whose expression is relatively constant, such as Lamin A/C. Alternatively, co-transfection with a plasmid containing a cellular marker, such as a CMV-driven EGFP-expression plasmid, luciferase, metalloprotease, BirA, β-galactosidase and the like may also be used to assess transfection efficiency. Cells which have been transfected with the siRNA molecules can then be identified by routine techniques such as immunofluorescence, phase contrast microscopy and fluorescence microscopy.

A viral-mediated delivery mechanism may also be employed to deliver siRNAs to cells in vitro and in vivo as described in Xia, H. et al. (2002) Nat Biotechnol 20(10): 1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501). Other methods of introducing siRNA molecules of the present invention to target cells, such as neural cells, epithelial cells, macrophages, and all other body cells, include a variety of art-recognized techniques including, but not limited to, calcium phosphate or calcium chloride co-precipitation, DEAE-dextran-mediated transfection, lipofection, or electroporation as well as a number of commercially available transfection kits (e.g., OLIGOFECTAMINE® Reagent from Invitrogen, Carlsbad, Calif.; LIPOFECTAMINE®™ 2000 from Invitrogen, Carlsbad, Calif.; I-FECT™ from Neuromics, Bloomington, Minn.; JetSI/DOPE (Avanti Polar Lipids, Alabaster, Ala.) (see, e.g. Sui, G. et al. (2002) Proc. Natl. Acad. Sci. USA 99:5515-5520; Calegari, F. et al. (2002) Proc. Natl. Acad. Sci. 99:14236-40; J-M Jacque, K. Triques and M. Stevenson (2002) Nature 418:435-437; and Elbashir, S. M et al. (2001) supra). Suitable methods for transfecting a target cell, e.g., a neuronal cell, a macrophage, an epithelial cell, can also be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals. The efficiency of transfection may depend on a number of factors, including the cell type, the passage number, the confluency of the cells as well as the time and the manner of formation of siRNA- or shRNA-liposome complexes (e.g., inversion versus vortexing). These factors can be assessed and adjusted without undue experimentation by one with ordinary skill in the art.

The siRNAs or shRNAs of the invention, may be introduced along with components that perform one or more of the following activities: enhance uptake of the siRNA, by the target cell, inhibit annealing of single strands, stabilize single strands, or otherwise facilitate delivery to the target cell and increase inhibition of flavivirus gene expression.

The siRNA may also be directly introduced into the target cell, or introduced extracellularly into a cavity, interstitial space, into the circulation of an organism, introduced orally, introduced nasally, introduced intracranially or may be introduced by bathing a cell or organism in a solution containing the siRNA. The siRNA may also be introduced into cells via topical application to a mucosal membrane or dermally. Vascular or extravascular circulation, the blood or lymph system, and the cerebrospinal fluid are also sites where the agents may be introduced.

As used herein, the term “vector” refers to a nucleic acid molecule capable of transporting another nucleic acid to which it has been linked. One type of vector is a “plasmid”, which refers to a circular double stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors”, or more simply “expression vectors.” In general, expression vectors of utility in recombinant DNA techniques are often in the form of plasmids. In the present specification, “plasmid” and “vector” are used interchangeably as the plasmid is the most commonly used form of vector. However, the invention is intended to include all other forms of expression vectors, such as viral vectors (e.g., replication defective retroviruses, lentiviruses, adenoviruses and adeno-associated viruses), which serve equivalent functions. In a embodiment, lentiviruses are used to deliver one or more siRNA molecule of the present invention to a cell.

Within an expression vector, “operably linked” is intended to mean that the nucleotide sequence of interest is linked to the regulatory sequence(s) in a manner which allows for expression of the nucleotide sequence (e.g., in an in vitro transcription/translation system or in a target cell when the vector is introduced into the target cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). Furthermore, the siRNAs may be delivered by way of a vector comprising a regulatory sequence to direct synthesis of the siRNAs of the invention at specific intervals, or over a specific time period. It will be appreciated by those skilled in the art that the design of the expression vector can depend on such factors as the choice of the target cell, the level of expression of siRNA desired, and the like.

The expression vectors of the invention can be introduced into target cells to thereby produce siRNA molecules of the present invention. In one embodiment, a DNA template, for example, a DNA template encoding flavivirus genes such as capsid, envelope, non-structural protein 3, untranslated regions or any combination thereof, may be ligated into an expression vector under the control of RNA polymerase III (Pol III), and delivered to a target cell. Pol III directs the synthesis of small, noncoding transcripts which 3′ ends are defined by termination within a stretch of 4-5 thymidines. In one embodiment, the DNA template does not include the flavivirus capsid gene. In another embodiment, the DNA template encodes the flavivirus capsid gene in combination with the envelope gene, non-structural protein 3 gene and/or untranslated regions. Accordingly, DNA templates may be used to synthesize, in vivo, both sense and antisense strands of siRNAs which effect RNAi (Sui, et al. (2002) PNAS 99(8):5515).

The expression vectors of the invention may also be used to introduce shRNA into target cells. The useful expression vectors also be inducible vectors, such as tetracycline (see, e.g., Wang et al. Proc Natl Acad Sci U.S.A. 100: 5103-5106, 2003) or ecdysone inducible vectors (e.g., from Invitrogen) known to one skilled in the art.

As used herein, the term “target cell” is intended to refer to any cell in the body, into which an siRNA molecule of the invention, including a recombinant expression vector encoding an siRNA of the invention, has been introduced. The terms “target cell” and “host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. In one embodiment, a target cell is a mammalian cell, e.g., a human cell.

It is known that depending upon the expression vector and transfection technique used, only a small fraction of cells may effectively uptake the siRNA molecule. In order to identify and select these cells, antibodies against a cellular target can be used to determine transfection efficiency through immunofluorescence. Typical cellular targets include those which are present in the host cell type and whose expression is relatively constant, such as Lamin A/C. Alternatively, co-transfection with a plasmid containing a cellular marker, such as a CMV-driven EGFP-expression plasmid, luciferase, metalloprotease, BirA, B-galactosidase and the like may also be used to assess transfection efficiency. Cells which have been transfected with the siRNA molecules can then be identified by routine techniques such as immunofluorescence, phase contrast microscopy and fluorescence microscopy.

Depending on the abundance and the life-time (or turnover) of the targeted protein, a knock-down phenotype, e.g., a phenotype associated with siRNA inhibition of the target flavivirus gene expression may become apparent after 1 to 3 days, or even later. In cases where no phenotype is observed, depletion of the protein may be observed by immunofluorescence or Western blotting. If the protein is still abundant after 3 days, cells can be split and transferred to a fresh 24-well plate for re-transfection. In one embodiment the depletion of the expression of the allele is monitored using RNA quantification techniquest capable of easily distinguishing the expression of the disease allele from the expression of healthy allele.

If no knock-down of the targeted protein is observed, it may be desirable to analyze whether the target mRNA was effectively destroyed by the transfected siRNA duplex. Two days after transfection, total RNA can be prepared, reverse transcribed using a target-specific primer, and PCR-amplified with a primer pair covering at least one exon-exon junction in order to control for amplification of pre-mRNAs. RT-PCR of a non-targeted mRNA is also needed as control. Effective depletion of the mRNA yet undetectable reduction of target protein may indicate that a large reservoir of stable protein may exist in the cell. Multiple transfection in sufficiently long intervals may be necessary until the target protein is finally depleted to a point where a phenotype may become apparent.

The dose of the siRNA will be in an amount necessary to effect RNA interference, e.g., post translational gene silencing (PTGS), of the particular target gene, thereby leading to inhibition of target gene expression or inhibition of activity or level of the protein encoded by the target gene. Assays to determine expression of the target allele, are known in the art. For example, reduced levels of target gene mRNA may be measured by in situ hybridization (Montgomery et al., (1998) Proc. Natl. Acad. Sci., USA 95:15502-15507) or Northern blot analysis (Ngo, et al. (1998)) Proc. Natl. Acad. Sci., USA 95:14687-14692). In one embodiment, target gene transcription is measured using quantitative real-time PCR (Gibson et al., Genome Research 6:995-1001, 1996; Heid et al., Genome Research 6:986-994, 1996).

Method of Treatment and/or Prevention

The active siRNAs of the present invention are administered in prophylactically or therapeutically effective amounts. A prophylactically or therapeutically effective amount means that amount necessary, at least partly, to attain the desired effect, or to delay the onset of, inhibit the progression of, or halt altogether, the onset or progression of the particular viral infection being treated. Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. It is usual generally that a maximum dose be used, that is, the highest safe dose according to sound medical judgment. It will be understood by those of ordinary skill in the art; however, that a lower dose or tolerable dose may be administered for medical reasons, psychological reasons or for virtually any other reasons.

In one aspect, the invention provides a method for preventing in a subject, an infectious disease or disorder, by administering to the subject one or more therapeutic agents, e.g., the siRNAs as described herein. For example, the siRNAs described herein may be used as antivirals to substantially reduce transmission of diseases transmitted by flaviviruses, including but not limited to West Nile virus, Japanese Encephalitis virus, St. Louis Encephalitis virus, Dengue virus. Subjects at risk for an infectious disease or disorder, can be identified by, for example, travel history, travel plans, lifestyle, immune state, pregnancy, old age or any known risk factors for an infectious disease or disorder.

Administration of a prophylactic agent can occur prior to the manifestation of symptoms characteristic of an infectious, disease or disorder, such that the infectious disease or disorder is prevented or, alternatively, delayed in its progression. Any mode of administration of the therapeutic agents of the invention, as described herein or as known in the art, including parenteral, intranasal or intracranial administration of the siRNAs of the instant invention, may be utilized for the prophylactic treatment of an infectious disease or disorder.

Formulations of the active compounds as described herein (e.g., an siRNA) may be administered to a subject at risk for an flavivirus-mediated disease or disorder, e.g., a viral disorder, such as disease mediated by West Nile virus, Japanese Encephalitis virus, St. Louis Encephalitis virus, Dengue virus, or another mosquito-transmitted disease or infection, or any other infectious agent, such as a virus, as a parenteral, intranasal or intracranial applied prophylactic to prevent transmission of a viral or bacterial disease or disorder, such as disease mediated by West Nile virus, Japanese Encephalitis virus, St. Louis Encephalitis virus, Dengue virus, or another mosquito-transmitted disease or infection. In one embodiment, the compositions comprising the siRNA and the carrier polymer may be administered prior to exposure to the infectious agent. In vitro experiments illustrate that the antiviral state induced by introduced duplex siRNAs can last for three weeks. Therefore, in one embodiment, an siRNA-based antiviral need not be applied before encounter with an infectious agent. Accordingly, in another embodiment, the prophylactic effect of the siRNA is prolonged, e.g., lasts for at least one week, in one embodiment two or more weeks. In another embodiment, the compositions comprising the siRNA may be administered, e.g., parenterally or intranasally, at intervals, e.g., one or more times per week, or one or more times per month, rather than directly prior to exposure to an infectious agent.

In another aspect, the invention provides a method for treating in a subject, an infectious disease or disorder, by administering to the subject one of more therapeutic agents, e.g., the siRNAs as described herein. For example, the siRNAs described herein may be used as antivirals administered to a subject infected by a flavivirus, for example WNV, JEV, Dengue virus, or any combination thereof, to substantially reduce viral load, viral shedding, virus mediated disease symptoms, further transmission of the virus, reactivation of the virus, reinfection of the subject with the same virus, infection of the subject with a different flavivirus or strain of flavivirus or any combination thereof.

The term “therapeutically effective amount” refers to an amount that is sufficient to effect a therapeutically or prophylactically significant reduction in production of infectious virus particles and reduction in viral shedding when administered to a typical subject who is either infected with flavivirus and at risk of spreading the virus or who is at risk of being infected with flavivirus. In aspects involving administration of an antiviral siRNA to a subject, typically the siRNA, formulation, or composition should be administered in a therapeutically effective amount.

Generally, the amount needed is less than the amount needed in antisense treatment applications (see, e.g., Bertrand et al. Biochemical and Biophysical Research Communications 296: 1000-1004, 2002). Antisense therapy has been used in human treatment methods and a skilled artisan may seek additional guidance in dosaging, for example, from publications such as “Results of a Pilot Study Involving the Use of an Antisense Oligodeoxynucleotide Directed Against the Insulin-Like Growth Factor Type I Receptor in Malignant Astrocytomas” by David W. Andrews, et al. in J. Clin Oncol, April 15: 2189-2200, 2001.

Generally, at intervals to be determined by the prophylaxis or treatment of pathogenic states, doses of active component will be from about 0.01 mg/kg per day to 1000 mg/kg per day. Small doses (0.01-1 mg) may be administered initially, followed by increasing doses up to about 1000 mg/kg per day. In the event that the response in a subject is insufficient at such doses, even higher doses (or effective higher doses by a different, more localized delivery route) may be employed to the extent patient tolerance permits. Multiple doses per day can be contemplated to achieve appropriate systemic levels of compounds.

Another aspect of the invention pertains to methods of modulating gene expression or protein activity, for example, cellular gene expression or activity and/or expression or activity of a gene or sequence of the flavivirus associated with flavivirus entry or replication, or viral gene expression or protein activity in order to treat an flavivirus infection or disorder. Accordingly, in an exemplary embodiment, the modulatory method of the invention involves contacting a cell with a therapeutic agent (e.g., one or more siRNAs, e.g., one or more siRNAs targeting a cellular gene or sequence and/or one or more siRNAs targeting a gene or sequence of an infectious agent, e.g., a viral gene or sequence), such that expression of the target gene or genes is prohibited. These methods can be performed in vitro, for example by culturing the cell, or in vivo, for example by administering the siRNA to a subject infected with flavivirus or at risk of infection with flavivirus.

The prophylactic or therapeutic pharmaceutical compositions of the invention can contain other pharmaceuticals, in conjunction with a vector according to the invention, when used to therapeutically treat flavivirus mediated disease, and can also be administered in combination with other pharmaceuticals used to treat flavivirus or symptoms of flavivirus mediated disease. For example, the prophylactic or therapeutic pharmaceutical compositions of the invention can also be used in combination with other pharmaceuticals which treat or alleviate symptoms of flavivirus infection or prevent secondary infections such as antibiotics used to prevent pneumonia and urinary tract infections, anticonvulsants for seizure control, antinausea medicants, mannitol, interferon-alpha, antibody therapy or any combination thereof. The prophylactic or therapeutic pharmaceutical compositions of the invention can be used in combination with practices used in supportive care of flavivirus infection, such as airway management, respiratory support, intravenous fluids, or any combination thereof.

A method of inhibiting expression of flavivirus mRNA, or mutant or variant thereof, comprising administering to a subject an effective amount of siRNA comprising a sense RNA strand and an antisense RNA strand, or a single RNA strand, wherein the sense and the antisense RNA strands, or the single RNA strand, form an RNA duplex, and wherein the RNA strand comprises a nucleotide sequence identical to a target sequence of about 19 to about 25 contiguous nucleotides in flavivirus mRNA, or an alternative splice form, mutant or cognate thereof, is degraded.

A method of preventing flavivirus mediated disease in a subject, comprising administering to a subject an effective amount of an siRNA comprising a sense RNA strand and an antisense RNA strand, or a single RNA strand, wherein the sense and the antisense RNA strands, or the single RNA strand, form an RNA duplex, and wherein the RNA strand comprises a nucleotide sequence identical to a target sequence of about 19 to about 25 contiguous nucleotides in flavivirus mRNA, or mutant or variant thereof.

The term “preventing” as used herein refers to preventing flavivirus infection in an individual susceptible for infection. Whether effective prevention is achieved can be tested using routine flavivirus detection methods including, but not limited to, IgM ELISA, IgG ELISA, IgA ELISA, blocking ELISA, IgG by indirect fluorescent antibody (IFA), microsphere immunoassay, Plaque Reduction Neutralization Test (PRNT), RT-PCR, Real Time RT-PCR, quantitative RT-PCR, TAQMAN® (Roche) assay, Nucleic Acid Sequence Based Amplification (NASBA; BioMerieux, Marcy l'Etoile, France) or any combination thereof, using blood, serum, cerebral spinal fluid or any combination thereof.

The invention further provides a method of treating flavivirus mediated disease in a subject comprising administering to the subject, such as a mammal, for example an equine, such as a horse, or a primate, such as a human, an effective amount of the siRNAs of the present invention comprising a sense RNA strand and an antisense RNA strand, or a single RNA strand, wherein the sense and an antisense RNA strands, or the single RNA strand, form an RNA duplex, and wherein the RNA strand comprises a nucleotide sequence identical to a target sequence of about 19 to about 25 contiguous nucleotides in flavivirus mRNA, or mutant or variant thereof.

In one embodiment, the siRNA used in the methods of the invention, are actively taken up by cells in vivo following intracranial administration, illustrating efficient in vivo delivery of the siRNAs used in the methods of the invention.

Other strategies for delivery of the siRNAs used in the methods of the invention, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the siRNAs or shRNAs of the invention, using a basic peptide by conjugating or mixing the siRNA with a basic peptide, e.g., a fragment of a TAT peptide, mixing with cationic lipids or formulating into particles.

In one embodiment, the dsRNA, such as siRNA or shRNA, is delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used.

In one embodiment, the siRNAs used in the methods of the invention, can be introduced into cells, e.g., cultured cells, which are subsequently transplanted into the subject by, e.g., transplanting or grafting, or alternatively, can be obtained from a donor (i.e., a source other than the ultimate recipient), and applied to a recipient by, e.g., transplanting or grafting, subsequent to administration of the siRNAs of the invention to the cells. Alternatively, the siRNAs of the invention can be introduced directly into the subject in such a manner that they are directed to and taken up by the target cells and regulate or promote RNA interference of the target flavivirus gene. The siRNAs of the invention may be delivered singly, or in combination with other siRNAs, such as, for example siRNAs directed to other viral strains or cellular genes associated with flavivirus entry or replication. The siRNAs of the invention may also be administered in combination with other pharmaceutical agents which are used to treat flavivirus infection.

The flavivirus targeting siRNAs are designed so as to maximize the uptake of the antisense (guide) strand of the siRNA into RNA-induced silencing complex (RISC) and thereby maximize the ability of RISC to target flavivirus mRNA for degradation. This can be accomplished by looking for sequences that has the lowest free energy of binding at the 5′-terminus of the antisense strand. The lower free energy would lead to an enhancement of the unwinding of the 5′-end of the antisense strand of the siRNA duplex, thereby ensuring that the antisense strand will be taken up by RISC and direct the sequence-specific cleavage of the flavivirus mRNA.

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double stranded fragments termed siRNAs. siRNAs are incorporated into a protein complex that recognizes and cleaves target mRNAs. RNAi can also be initiated by introducing nucleic acid molecules, e.g., synthetic siRNAs, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease may be of at least 30%, 40%, 50%, 60%, 70%, 80%, 90%, 95% or 99% or more as compared to the expression of a target gene or the activity or level of the protein encoded by a target gene which has not been targeted by an siRNA.

The target gene or sequence of the siRNA is designed to be substantially homologous to the target sequence, or a fragment thereof. As used herein, the term “homologous” is defined as being substantially identical, sufficiently complementary, or similar to the target flavivirus mRNA, or a fragment thereof, to effect RNA interference of the target. In addition to native RNA molecules, RNA suitable for inhibiting or interfering with the expression of a target sequence include RNA derivatives and analogs. In one embodiment, the siRNA is identical to its target allele so as to prevent its interaction with the normal allele.

The siRNAs used in the methods of the invention typically target only one sequence. In one embodiment, a mixture of siRNAs designed to inhibit expression of one or more flavivirus sequences are used in combination. Each of the siRNAs, can be screened for potential off-target effects may be analyzed using, for example, expression profiling. Such methods are known to one skilled in the art and are described, for example, in Jackson et al. Nature Biotechnology 6:635-637, 2003. In addition to expression profiling, one may also screen the potential target sequences for similar sequences in the sequence databases to identify potential sequences which may have off-target effects. For example, according to Jackson et al. (Id.) 15, or perhaps as few as 11 contiguous nucleotides, of sequence identity are sufficient to direct silencing of non-targeted transcripts. Therefore, one may initially screen the proposed siRNAs to avoid potential off-target silencing using the sequence identity analysis by any known sequence comparison methods, such as Basic Local Alignment Search Tool (BLAST) from NCBI (U.S. National Institutes of Health information database).

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues may be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatizes with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated.

In one embodiment, siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology.

Various aspects of the invention are described in further detail in the following subsections:

Short Interfering RNAs (siRNAs) of the Invention

In one embodiment, the siRNA useful in the methods of treatment and prevention of flavivirus infection are described above.

Other siRNAs useful in preventing or treating flavivirus according to the methods of the present invention may be readily designed and tested. Accordingly, the present invention also relates to siRNA molecules of about 15 to about 30 or about 15 to about 28 nucleotides in length, which are homologous to an flavivirus gene. In one embodiment, the siRNA molecules have a length of about 19 to about 25 nucleotides. In another embodiment, the siRNA molecules have a length of about 19, 20, 21, or 22 nucleotides. The siRNA molecules of the present invention can also comprise a 3′ hydroxyl group. The siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In one embodiment, at least one strand of the RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment the RNA molecule is double stranded, one strand has a 3′ overhang and the other strand can be blunt-ended or have an overhang. In the embodiment in which the RNA molecule is double stranded and both strands comprise an overhang, the length of the overhangs may be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

In one embodiment, the siRNA of the present invention comprises two molecules where the sense RNA strand comprises one RNA molecule, and the antisense RNA strand comprises one RNA molecule; or the sense and antisense RNA strands forming the RNA duplex may be covalently linked by a single-stranded hairpin. In another embodiment, the siRNA is comprised of non-nucleotide material. In yet another embodiment, the sense and antisense RNA strands of the siRNA may be stabilized against nuclease degradation. The siRNA may contain one or two 3′ overhangs comprising from 1 to about 6 nucleotides each. Alternatively, the 3′ overhang is comprised of a dinucleotide of dithymidylic acid (TT) or diuridylic acid (uu). In yet another embodiment, the 3′ overhang is stabilized against nuclease degradation.

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

Design and Preparation of siRNA Molecules

Synthetic siRNA molecules, including shRNA molecules, of the present invention can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

The targeted region of the siRNA molecule of the present invention can be selected from a given target gene sequence, e.g., an envelope glycoprotein or a DNA binding protein, beginning from about 25 to 50 nucleotides, from about 50 to 75 nucleotides, or from about 75 to 100 nucleotides downstream of the start codon. Nucleotide sequences may contain 5′ or 3′ UTRs and regions nearby the start codon. One method of designing a siRNA molecule of the present invention involves identifying the 23 nucleotide sequence motif AA(N19)TT (SEQ ID NO: 138) (where N can be any nucleotide) and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search may be extended using the motif NA(N21), where N can be any nucleotide. In this situation, the 3′ end of the sense siRNA may be converted to TT to allow for the generation of a symmetric duplex with respect to the sequence composition of the sense and antisense 3′ overhangs. The antisense siRNA molecule may then be synthesized as the complement to nucleotide positions 1 to 21 of the 23 nucleotide sequence motif. The use of symmetric 3′ TT overhangs may be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but not limited to the NCBI, BLAST, Derwent and GenSeq as well as commercially available oligosynthesis companies such as Oligoengine®, may also be used to select siRNA sequences against EST libraries to ensure that only one gene is targeted.

The siRNAs as described herein including the flavivirus targeting siRNA can be administered to individuals to treat flavivirus mediated disease. In conjunction with such treatment, pharmacogenomics (i.e., the study of the relationship between an individual's genotype and that individual's response to a foreign compound or drug) may be considered. Differences in metabolism of therapeutics, including siRNAs, can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer one or more therapeutic siRNAs as described herein as well as tailoring the dosage and/or therapeutic regimen of treatment with an siRNA targeting a flavivirus gene.

For example, in one embodiment, before administering the siRNA to an individual, the target sequence of the flavivirus viral strain harbored by the individual may be analyzed for any potential gene variations, such as polymorphisms or mutations, in the region against which the siRNA is targeted. For example, one may sequence the UL29 gene from the strain harbored by the individual. If one or more mutations or a polymorphisms is detected, the siRNA may be modified to target the specific mutant or polymorphic form of the target.

### Pharmaceutical Compositions

The invention also provides a pharmaceutical composition comprising at least one siRNA and a pharmaceutically acceptable carrier, wherein the siRNA comprises a sense RNA strand and an antisense RNA strand, or a single RNA strand, wherein the sense and the antisense RNA strands, or the single RNA strand, form an RNA duplex, and wherein the RNA strand comprises a nucleotide sequence identical to a target sequence of about 19 to about 25 contiguous nucleotides in flavivirus mRNA, or an alternative splice form, mutant or cognate thereof.

A pharmaceutically acceptable carrier refers to generally available and known pharmaceutical carriers and diluents. The formulation of such compositions is well known to persons skilled in this field. Suitable pharmaceutically acceptable carriers and/or diluents include any and all solvents, dispersion media, fillers, solid carriers, aqueous solutions, coatings, antibacterial and anti fungal agents, isotonic, and absorption enhancing or delaying agents, activity enhancing or delaying agents and the like. The use of such media and agents for pharmaceutically active substances is well known in the art, and it is described, by way of example, in Remington's Pharmaceutical Sciences, 18th Edition, Mack Publishing Company, Pennsylvania, USA. Except insofar as any conventional carrier and/or diluent is incompatible with the active ingredient, use thereof in the pharmaceutical compositions of the present invention is contemplated. Supplementary active ingredients including agents having antiviral or antimicrobial activity can also be incorporated into the compositions of this invention.

The active compositions of the present invention may include classic pharmaceutical preparations. Administration of these compositions according to the present invention will be via any common route so long as the target tissue is available via that route. The routes of administration will vary, naturally, with the location and nature of the infection, and the dosage required for prophylactic or therapeutic efficacy. The routes of administration include, e.g., intradermal, transdermal, transmucosal, parenteral, intracranial, intravenous, intramuscular, intranasal, intracerebrospinal, subcutaneous, percutaneous, intratracheal, intraperitoneal, intratumoral, perfusion, lavage, direct injection, and oral administration and formulation. In the present invention, intracranial, intranasal or intravenous administration are exemplary embodiments. Administration may be by injection or infusion. The methods of this invention, generally speaking, may be practiced using any mode of administration that is medically acceptable, meaning any mode that produces prophylactic or therapeutic levels of the active component of the invention without causing clinically unacceptable adverse effects.

For methods of performing intracranial administration, please see Kruse et al. (J. Neuro-Oncol., 19:161-168, 1994), specifically incorporated by reference. Such compositions would normally be administered as pharmaceutically acceptable compositions.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Parenteral modes of administration for the present invention include intravenous, intracranial, intramuscular, intradermal, subcutaneous, and oral (e.g., inhalation) administrations. Solutions or suspensions used for parenteral, intracranial, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

The pharmaceutical compositions as described herein are in one embodiment capable of crossing the blood-brain barrier (BBB). For example, the composition may comprise a brain targeting agent or moiety, such as an anti-insulin receptor antibody (Coloma et al., (2000) Pharm Res 17:266-74), anti-transferrin receptor antibodies (Zhang and Pardridge, (2001) Brain Res 889:49-56) or activated T-cells (Westland et al., (1999) Brain 122:1283-91). Alternatively, techniques resulting in modification of the vasculature by the use of vasoactive peptides such as bradykinin or other techniques such as osmotic shock (reviewed in Begley, (1996) J Pharm Pharmacol 48:136-46; Neuwelt et al., (1987) Neurosurgery 20:885-95; Kroll et al., (1998) Neurosurgery 43:879-86; Temsamani et al., (2000) Pharm Sci Technol Today 3:155-162) may be employed. Further compositions include those in U.S. Pat. Nos. 6,372,250; 5,130,129; 5,004,697; and 4,902,505; U.S. Pat. App. Nos 2005/0085419, 2005/0042298, 2005/0042227, 2005/0026823, 2004/0102369, 2004/0101904, and 2003/0129186; Int'l Pat. App. Nos. WO 04/050016, WO 01/07084, WO 99/00150, WO 98/22092 and WO 92/22332.

The blood-brain barrier targeting agent may be any of the known vectors that undergo receptor mediated transport across the BBB via endogenous peptide receptor transport systems localized in the brain capillary endothelial plasma membrane, which forms the BBB in vivo. In one embodiment, targeting agents include insulin, transferrin, insulin-like growth factor (IGF), leptin, low density lipoprotein (LDL), and the corresponding peptidomimetic monoclonal antibodies that mimic these endogeneous peptides. Peptidomimetic monoclonal antibodies bind to exofacial epitopes on the BBB receptor, removed from the binding site of the endogenous peptide ligand, and “piggyback” across the BBB via the endogenous peptide receptor-mediated transcytosis system. Peptidomimetic monoclonal antibodies are species specific. For example, the OX26 murine monoclonal antibody to the rat transferrin receptor is used for drug delivery to the rat brain (Pardridge et al. 1991. J Pharmacol Exp Ther 256:66-70). The OX26 antibody to the rat transferrin receptor does not work in other species, including mice (Lee et al. 2000. J Pharmacol. Exp Ther 292: 1048-1052). Accordingly, the OX26 antibody to the rat transferrin receptor would not be used in humans. The OX-26 monoclonal antibody, as described in the following examples, is a suitable transferrin receptor targeting agent for rats. Monoclonal antibodies to the human insulin receptor (HIR) are typically used for delivering the pharmaceutical composition to the human brain. In one embodiment, “humanized” monoclonal antibodies are used, and one does not use the original mouse form of the antibody. Exemplary, humanized monoclonal antibodies to the human insulin receptor that are particularly well-suited for use in the present invention are described in detail in U.S. Pat. App. No. 2004/0101904, the contents of which application are hereby specifically incorporated by reference. Other possible targeting agents include the rat 8D3 or rat RI7-217 monoclonal antibody to the mouse transferrin receptor for drug delivery to mouse brain (Lee et al. 2000. J Pharmacol Exp Ther 292: 1048-1052), or murine, chimeric or humanized antibodies to the human or animal transferrin receptor, the human or animal leptin receptor, the human or animal IGF receptor, the human or animal LDL receptor, the human or animal acetylated LDL receptor.

In one embodiment, the route of administration for pharmaceutical compositions of the present invention that are targeted to the brain is intravenous. Suitable carriers include saline or water buffered with acetate, phosphate, TRIS or a variety of other buffers, with or without low concentrations of mild detergents, such as one from the Tween series of detergents.

In certain embodiments, the pharmaceutical compositions may be delivered by intranasal sprays, inhalation, and/or other aerosol delivery vehicles. For aerosol delivery vehicles, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch. Methods for delivering genes, nucleic acids, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroethylene support matrix is described in U.S. Pat. No. 5,780,045 (specifically incorporated herein by reference in its entirety). As previously shown, intranasal administration (Mathison et al, J. Drug Target, 5 (6):415-441 (1998); Chou et al, Biopharm Drug Dispos. 18 (4):335-46 (1997); Draghia et al, Gene Therapy 2:418-423 (1995)) may enable the direct entry of viruses and macromolecules into the CSF or CNS.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or vaginal suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery. Suitable formulations for transdermal and transmucosal administration include solutions, suspensions, gels, lotions and creams as well as discrete units such as suppositories and microencapsulated suspensions. Other delivery systems can include sustained release delivery systems which can provide for slow release of the active component of the invention, including sustained release gels, creams, suppositories, or capsules. Many types of sustained release delivery systems are available. These include, but are not limited to: (a) erosional systems in which the active component is contained within a matrix, and (b) diffusional systems in which the active component permeates at a controlled rate through a polymer.

In another embodiment, pharmaceutical compositions may be delivered by ocularly via eyedrops.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, cherry, grape or orange flavoring.

The siRNA of the invention can be incorporated into pharmaceutical or antiviral compositions suitable for administration. Such compositions typically comprise the siRNA targeting an flavivirus gene, and a pharmaceutically acceptable carrier as defined herein. Supplementary active compounds can also be incorporated into the compositions.

One pharmaceutical or antiviral composition according to the present invention comprises siRNA targeting flavivirus sequences. In one embodiment, Cacipacore virus, Koutango virus, Murray Valley encephalitis virus, St. Louis Encephalitis virus, Alfuy virus, Kunjin virus, Yaounde virus or any combination thereof are targeted. In one embodiment, WNV, JEV, Dengue virus or any combination thereof are targeted. In another embodiment, a gene that is essential to flavivirus is targeted. In one embodiment, sequence that is shared among 2, 3, 4, 5, 6, 7, 8, 9 or 10 species of flavivirus is targeted. In one embodiment, at least one of the targets include, but are not limited to, mRNA encoding C, E, and NS3 genes and untranslated regions of the flavivirus mRNA. In one embodiment, the pharmaceutical composition comprises SEQ ID NOS: 1-95 from Table 1 or any combination thereof.

A pharmaceutical composition of the invention is formulated to be compatible with its intended route of administration. Generally, the compositions of the instant invention are introduced by any standard means, with or without stabilizers, buffers, and the like, to form a pharmaceutical composition. For use of a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the like.

In one embodiment, the invention features the use of the compounds of the invention in a composition comprising surface-modified liposomes containing poly (ethylene glycol) lipids (PEG-modified, or long-circulating liposomes or stealth liposomes). In another embodiment, the invention features the use of compounds of the invention covalently attached to polyethylene glycol. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). The long-circulating compositions enhance the pharmacokinetics and pharmacodynamics of therapeutic compounds, such as DNA and RNA, particularly compared to conventional cationic liposomes which are known to accumulate in tissues of the MPS (Liu et al., J. Biol. Chem. 1995, 42, 2486424870; Choi et al., International PCT Publication No. WO 96/10391; Ansell et al., International PCT Publication No. WO 96/10390; Holland et al., International PCT Publication No. WO 96/10392). Long-circulating compositions are also likely to protect drugs from nuclease degradation to a greater extent compared to cationic liposomes, based on their ability to avoid accumulation in metabolically aggressive MPS tissues such as the liver and spleen.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to hepatocytes) can also be used as pharmaceutically acceptable carriers. These can be prepared according to methods known to those skilled in the art, for example, as described in U.S. Pat. No. 4,522,811 U.S. Pat. No. 5,643,599, the entire contents of which are incorporated herein.

Liposomal suspensions (including liposomes targeted to macrophages containing, for example, phosphatidylserine) can also be used as pharmaceutically acceptable carriers. These can be prepared according to methods known to those skilled in the art, for example, as described in U.S. Pat. No. 4,522,811 U.S. Pat. No. 5,643,599, the entire contents of which are incorporated herein. Alternatively, the therapeutic agents of the invention may be prepared by adding a poly-G tail to one or more ends of the siRNA for uptake into target cells. Moreover, siRNA may be fluoro-derivatized and delivered to the target cell as described by Capodici, et al. (2002) J. Immuno. 169(9):5196.

Sterile injectable solutions can be prepared by incorporating the siRNA in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, in one embodiment, methods of preparation are vacuum drying and freeze-drying which yields a powder of the active ingredient plus any additional desired ingredient from a previously sterile-filtered solution thereof.

The siRNAs of the invention can be inserted into vectors. These constructs can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the vector can include the siRNA vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies typically within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of an siRNA (i.e., an effective dosage) ranges from about 0.001 to 3000 mg/kg body weight, in one embodiment, about 0.01 to 2500 mg/kg body weight, in one embodiment, the amount is about 0.1 to 2000 mg/kg body weight, and in another embodiment, the amount is about 1 to 1000 mg/kg, 2 to 900 mg/kg, 3 to 800 mg/kg, 4 to 700 mg/kg, or 5 to 600 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an siRNA can include a single treatment or, in one embodiment, can include a series of treatments.

For example, a subject is treated with an siRNA in the range of between about 0.1 to 20 mg/kg body weight, one time per week for between about 1 to 10 weeks, in one embodiment, between 2 to 8 weeks, in another embodiment, between about 3 to 7 weeks, and yet in another embodiment, for about 4, 5, or 6 weeks. It will also be appreciated that the effective dosage of siRNA used for treatment may increase or decrease over the course of a particular treatment. Changes in dosage may result and become apparent from the results of diagnostic assays as described herein.

It is understood that appropriate doses of the siRNAs or shRNAs, depend upon a number of factors within the ken of the ordinarily skilled physician, veterinarian, or researcher. The dose(s) of the agent will vary, for example, depending upon the identity, size, and condition of the subject or sample being treated, further depending upon the route by which the composition is to be administered, if applicable, and the effect which the practitioner desires the siRNA to have upon flavivirus.

The pharmaceutical compositions can be included in a container, pack, or dispenser together with instructions for administration.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications and publications cited herein are incorporated herein by reference.

### EXAMPLES

### Example 1

### Materials and Methods

### Cells and Viruses

Baby hamster kidney (BHK21), the mouse neuronal cell line (Neuro 2a) and Vero cell lines were all obtained from ATCC and maintained in DMEM with 10% FCS. The Nakayama strain of JEV and B956 strain of WNV were obtained from ATCC, grown and plaque titrated using BHK21 cells for in vitro studies. Lethal dose (LD50) for both viruses was determined by inoculating serial dilutions of infected mouse brain lysates into groups of mice as described in²⁴.

siRNA Sequence and Viral Vector to Express shRNA

The sequence of the siRNAs designed to target the envelope gene were as in Table 3. To generate a lentiviral vector to express shFvE^{J}, two complementary oligonucleotides incorporating FvE^{J} sequence were synthesized as a 21-nucleotide inverse repeat separated by a 9 nucleotide loop sequence and cloned in front of the U6 promoter in the lentiviral vector lentilox pLL3.7 as described by Rubinson et al¹⁶. A control vector targeting the luciferase gene was also similarly generated using a published sequence (nt 155-173)²⁵. Lentiviral stocks were generated by co-transfection of the lentiviral vector along the with helper plasmids pHR′8.9ΔVPR (core protein) and either pCMV-VSV-G or pLTR-RVG (envelope). After 48 h, supernatants were filtered using a 0.45 um membrane filter (Millipore), aliquoted and stored at −70° C. Concentrated virus preparations were made by ultrapelleting the supernatants in a SW28 rotor at 25,000 rpm for 1 hr. The virus was suspended in PBS for 3-4 h, aliquoted and stored at −70° C.

TABLE 3

| | Target | Seq | Sense | Seq | Antisense | Seq |
|---|---|---|---|---|---|---|
| | Sequence | ID | strand | ID | strand | ID |
| FvE^{J} | GGATGTGGACTTTTCGG | 101 | GGAUGUGGACU | 11 | UCCCGAAA | 12 |
| | GA JEV nt 1287-1305 | | UUUCGGGA | | AGUCCACA | |
| | | | | | UCC | |
| FvEw | GGCTGCGGACTGTTTGG | 102 | GGCUGCGGACU | 13 | UUCCAAAC | 14 |
| | AA WNV nt 1287-130 | | GUUUGGAA | | AGUCCGCA | |
| | | | | | GCC | |
| FvE^{J}w | GGGAGCATTGACACAT | 103 | GGGAGCAUUGA | 15 | UGCACAUG | 16 |
| | GTGCA JEV nt 1307-1328 | | CACAUGUGCA | | UGUCAAUG | |
| | | | | | CUCCC | |

Lentiviral stocks were titrated by inoculating serial dilutions on 293T (when pseudotyped with VSV-G) or Neuro 2a cells (when pseudotyped with the RV-G) and determining GFP expression by flow cytometry 2 days later and expressed as transduction units (TU)/ml.

Cell Lines Stably Expressing shRNA and JEV Challenge

BHK21 or Neuro 2a cells were spin infected with lentivirus for 2 hr at 2400 rpm (moi of 10) in DMEM media containing 10% FCS and 8 μg/ml of polybrene. After 2 h further incubation at 37° C., fresh medium was added to the cells. After 2-3 days of culture, the transduction efficiency was ascertained on the basis of GFP expression (nearly 100% in both cell lines). Cells were challenged with JEV or WNV at different multiplicities of infection (moi). At different times post infection, the cells were stained with a JEV-specific antibody (ATCC) or WNV-envelope specific monoclonal antibody (Chemicon International) followed by a phycoerythrin-conjugated goat anti-mouse polyclonal antibody (DakoCytomation) and examined by flow cytometry.

Northern Blot to Detect shRNA and Viral RNA Degradation

For Northern Blot analysis, 5 μg of total cellular RNA, purified from by the RNEASY® mini kit (Qiagen), were run on a 1% denaturing agarose gel, transferred to a positively charged nylon membrane (BRIGHTSTAR®-plus, Ambion) and probed using the NORTHERNMAX™ protocol (Ambion). The JEV probe corresponded to the NS4b gene product of JEV, RT PCR amplified from JEV-infected BHK21 cellular RNA. The DECATEMPLATE™-beta-actin-probe (Ambion) was used for probing the β-actin mRNA that served as the loading control. The probes were labeled with ³²P dATP using the DECA prime II random prime labeling kit (Ambion), purified by NucAway spin columns (Ambion). Production of siRNA in lentivirus-transduced cells was analyzed by modified northern blot designed to capture small RNAs efficiently as described earlier²⁶.

siRNA Transfection

Neuro 2a cells were seeded in 6-well plates at 1×10⁵ per well for 12 to 16 hours before transfection. Lipid-siRNA complexes were prepared by incubating 200 nM of indicated siRNA with LIPOFECTAMINE® 2000 (Invitrogen), iFect (Neuromics Inc) or JetSI/DOPE (Avanti Polar Lipids, Inc, Alabaster, Ala.) formulations in the appropriate complexation volume as recommended by the manufacturer. Lipid-siRNA complexes were added to the wells in a final volume of 1 ml DMEM cell culture medium. After incubation for 6 h, cells were washed, and reincubated in DMEM media containing 10% FCS, and infected with flaviviruses 24 h post transfection.

### RT PCR and ELISA to Detect Interferon Inducible Genes and Serum Interferon Levels

Total RNA was isolated from homogenized mouse brain tissue with TRIzol Reagent (Invitrogen). A total of 5 μg from each sample was reverse transcribed using the REACTIONREADY™ first strand cDNA synthesis kit (SuperArray Bioscience Corporation, Frederick, Md.) according to the manufacturer's instruction. Following reverse transcription, the samples were processed for PCR using the MultiGene-12 reverse transcriptase-PCR profiling kit for mouse interferon response genes (SuperArray, Bioscience Corporation) according to the manufacturer's instruction. The PCR program consisted of an initial incubation at 95° C. for 15 min to denature the samples followed by 30 cycles of 95° C. for 30 s, 55° C. for 30 s, and 72° C. for 45 s. After completion of PCR, 10 μl of each sample was separated by agarose gel electrophoresis and stained and scanned as a digital image using a CCD camera. The PCR gene products were quantified by NIH Image J (version 1.32j) software. Values obtained for the test samples were normalized with respect to the GAPDH control and divided by the normalized values obtained with the brain sample from untreated mice to determine the fold increases in mRNA levels for each of the genes. IFN levels in serum and brain samples were quantified by using a sandwich mouse type I IFN detection ELISA kit from PBL Biomedical, according to the manufacturer's instructions.

### Mouse Infection

Balb/c mice (Jackson laboratory, Bar Harbor, Me.) aged 4-6 weeks were used for all in vivo experiments. All mouse infection experiments were done in a biosafety level 3 animal facility at the CBR Institute for Biomedical Research and had been approved by the institutional review board. For experiments using lentiviruses, mice were inoculated intracranially with different doses of lentivirus in 5 μl of PBS through the bregma (4 mm deep vertically into the brain using a Hamilton syringe fitted with a 30 gauge needle) at different times before the flaviviral challenge. The mice were subsequently challenged with different doses of JEV or WNV by intracranial inoculation through the bregma at the same spot as described above. For experiments using siRNA, siRNAs were complexed with iFect (Neuromics, Inc) or JetSI/DOPE (Avanti Polar Lipids, Inc, Alabaster, Ala.) according to the manufacturer's instruction. Intracranial injections of siRNA/lipid complexes and flaviviral challenge were done as described earlier.

Mouse tissue preparation: Mice were euthanized by anesthesia and brains removed and used in various experiments. For detection of neuronal cell infection by flow cytometry, freshly isolated brain specimens were used to make single cell suspension by gently teasing with the back of a syringe piston. For virus titrations, brain tissues were homogenized in HBSS-BSA (10% [wt/vol]) followed by repeated passage through a syringe fitted with a 29 gauge needle for at least 20 times at 4° C. to release all intracellular virus. Viral titrations were done as described earlier. In some experiments, the same mouse brain homogenates were inoculated on Neuro 2a cells, cultured for 5 days and examined by flow cytometry for viral antigen expression. For histology, the brain samples were fixed in 10% neutral buffered formalin, embedded in paraffin and 6 μm horizontal sections were stained with hematoxylin and eosin.

### Results and Discussion

The mosquito-borne flaviviruses such as the Japanese encephalitis (JE) and West Nile (WNV) viruses are among the most important examples of emerging and resurging pathogens. For example, after it was first introduced in the US in New York 1999, WNV rapidly spread throughout the continental US causing large outbreaks of disease with significant morbidity and mortality^{2,3}. Both WNV and JE viruses can cause a devastating acute neurological illness with up to 30% mortality and permanent neurological disabilities in the survivors⁴⁻⁶. Once the virus invades the central nervous system (CNS), the course of infection is very rapid, suggesting that success in developing antiviral treatment modalities would hinge on the ability to reduce the viral load early in the infection. Moreover, infections by diverse neurotropic flaviviruses are clinically indistinguishable, which makes it important to develop broad-based therapeutic approaches that are effective against multiple viruses within and across species. RNA interference (RNAi) has emerged as a powerful tool for gene silencing with a potential for therapeutic use in viral infections⁷⁻⁹. Several studies have demonstrated that the CNS is amenable to RNAi¹⁰⁻¹³. Here we explore the feasibility of using siRNA targeting conserved viral sequences, to inhibit multiple flaviviral encephalitides.

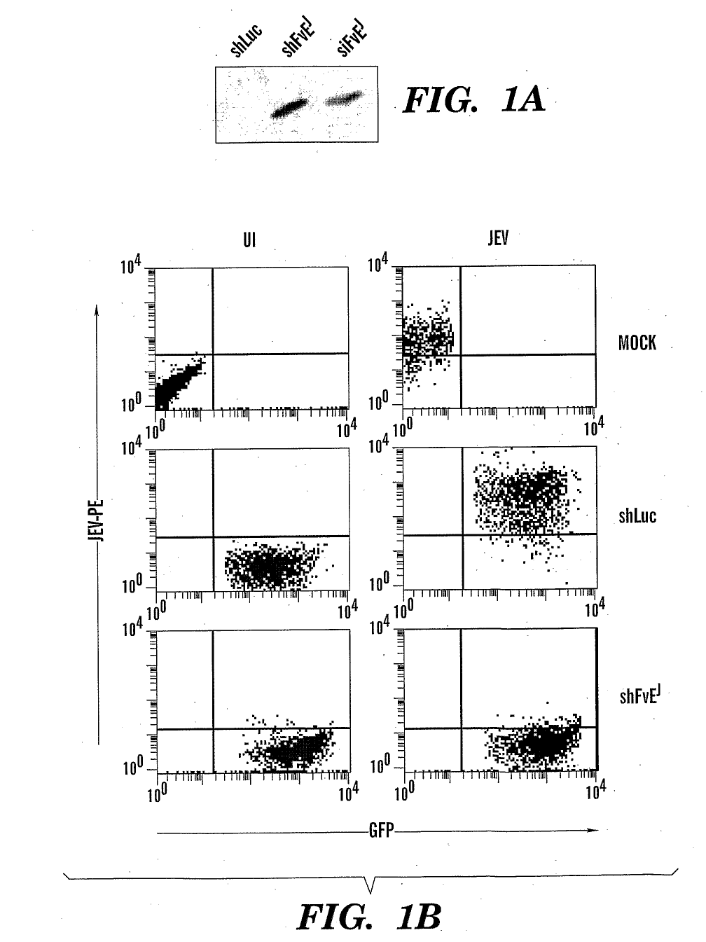

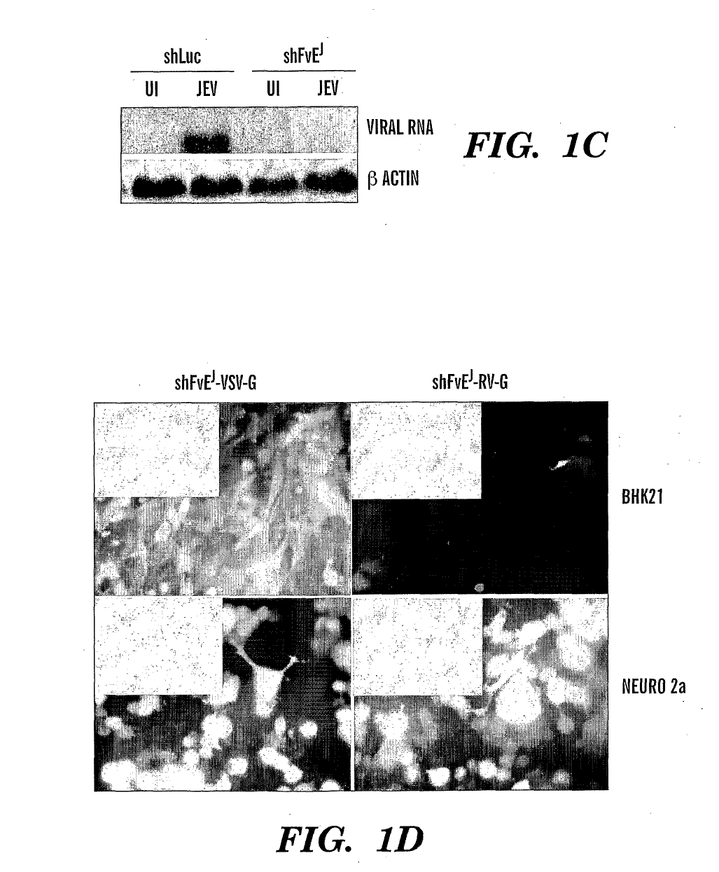

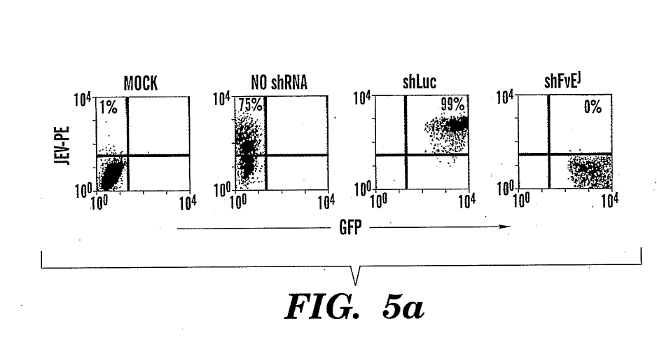

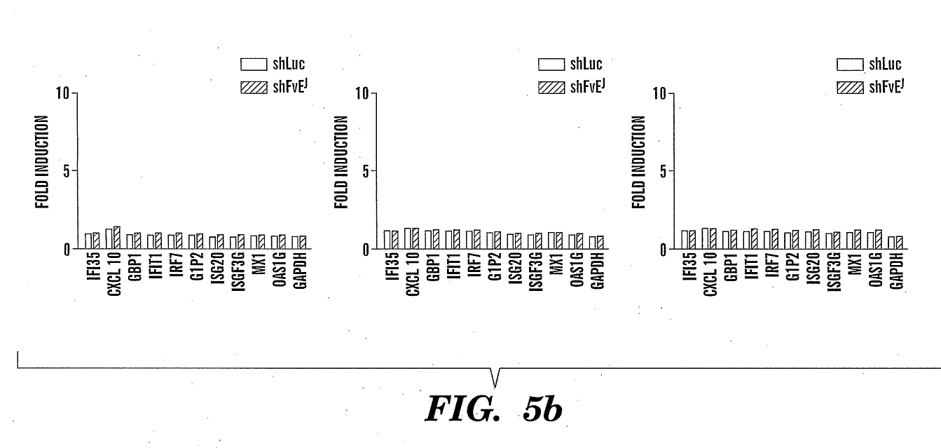

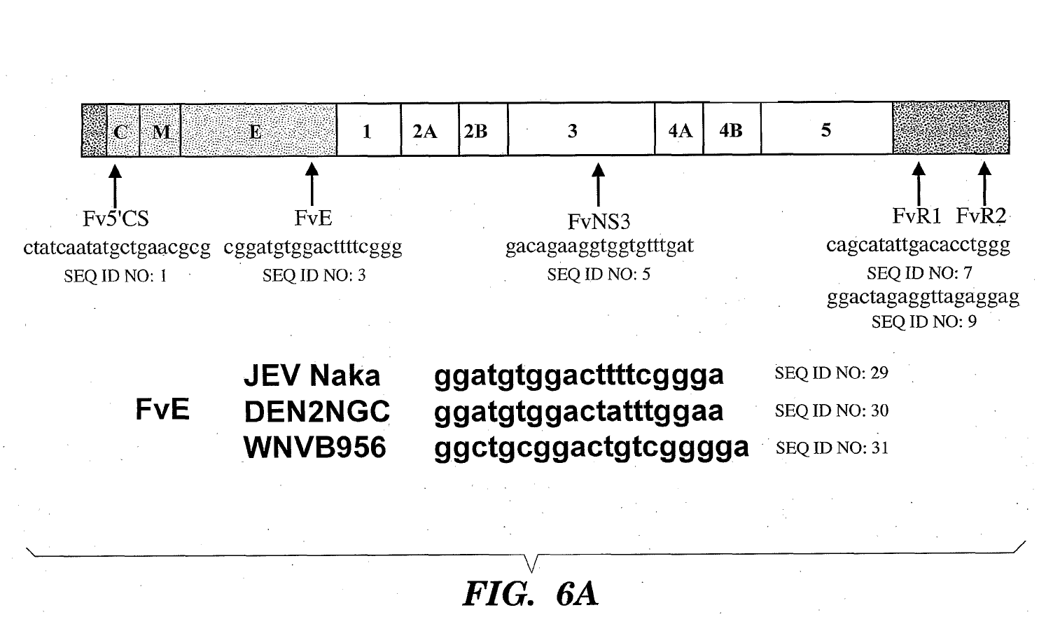

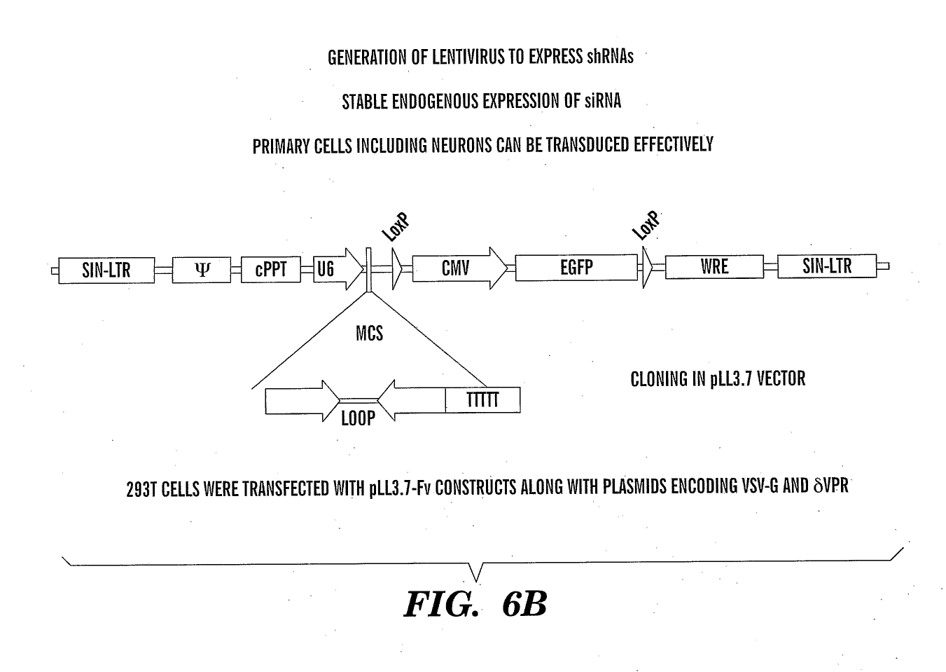

Flaviviruses are small (40-60 nm) enveloped viruses with a single-stranded positive-sense RNA genome that is approximately 11 kb long. The genome encodes for a single polyprotein which is processed into 3 structural and 7 non-structural proteins14,15. In initial studies, we compared the silencing ability of five synthetic short interfering RNAs (siRNAs) targeting different regions of the JEV genome and found that a siRNA that targets the envelope gene (FvEJ, nt 1287-1305 of the genomic RNA) afforded a robust protection against JEV infection in cell lines. Moreover, this sequence is completely conserved among all sequenced wild type JEV isolates (see e.g. comparisons in FIG. 6A). Since the siRNA effect diminishes over time in cell lines because of dilution with cell division, to follow the kinetics of protection, we cloned the sequence as a U6 promoter driven template for short hairpin RNA (shRNA) in the lentiviral vector pLL3.716. This vector also contains a green fluorescent protein (GFP) gene under the control of the Cytomegalovirus (CMV) promoter, which allows easy monitoring of transduced cells. When baby hamster kidney (BHK21) cell line was transduced with Vesicular stomatitis virus glycoprotein (VSV-G)-pseudotyped lentiviruses encoding FvEJ (shFvEJ) or the control luciferase shRNA (shLuc), nearly 100% of the cells were transduced as indicated by GFP expression. However, FvEJ-specific short 21 nt RNA was detected by Northern blot analysis only in cells stably transduced with shFvEJ, but not shLuc (FIG. 1a). To test the ability of the shRNA to inhibit viral replication, the transduced BHK21 cells were infected with JEV and 60 h later, the extent of infection was assessed by flow cytometry after staining with a JEV-specific antibody (ATCC). The high degree of infection seen in the mock- and shLuc-transduced cells was nearly abrogated with shFvEJ transduction (FIG. 1b). Decrease in the steady state levels of viral RNA in the shFvEJ-transduced cells was also confirmed by Northern analysis using a JEV-specific cDNA probe (FIG. 1c). The antiviral effect of FvEJ shRNA was not due to the induction of an interferon (IFN) response because shFvEJ was also able to inhibit viral replication in Vero cells that lack type I IFN genes17 (FIG. 5). Moreover, IFN-responsive genes were not upregulated in shFvEJ- compared to shLuc-transduced cells (FIG. 5). Thus, shFvEJ effectively inhibits JEV replication by RNAi-mediated degradation of viral RNA.

Pseudotyping the lentivirus with the neurotropic Rabies virus glycoprotein (RV-G) instead of the conventionally used VSV-G allows retrograde axonal transport to distal neurons and results in more extensive spread of the transduced genes 18. Moreover, RV-G pseudotyping may also allow neuronal cell-specific targeting, which could be an advantage with JEV, which like Rabies virus, preferentially targets neuronal cells in vivo. Thus, we tested lentiviruses pseudotyped with either VSV-G or, RV-G for their ability to deliver shRNA to non-neuronal or neuronal cells. Indeed, whereas the VSV-G pseudotyped lentivirus uniformly transduced both BHK21 and the mouse neuroblastoma cell line Neuro 2a, RV-G pseudotyping allowed transduction exclusively of Neuro 2a, but not BHK21 cells (FIG. 1d). Further, the RV-G-pseudotyped shFvEJ exhibited a more potent antiviral activity compared to the corresponding VSV-G pseudotyped lentivirus in that, it abrogated JEV infection in Neuro 2a even at an moi of 50 (highest dose tested) while the protection offered by VSV-G pseudotyped shFvEJ diminished at mois higher than 25. This may be due to differences in the respective receptor density, enabling better entry of RV-G pseudotyped virus in neuronal cells.

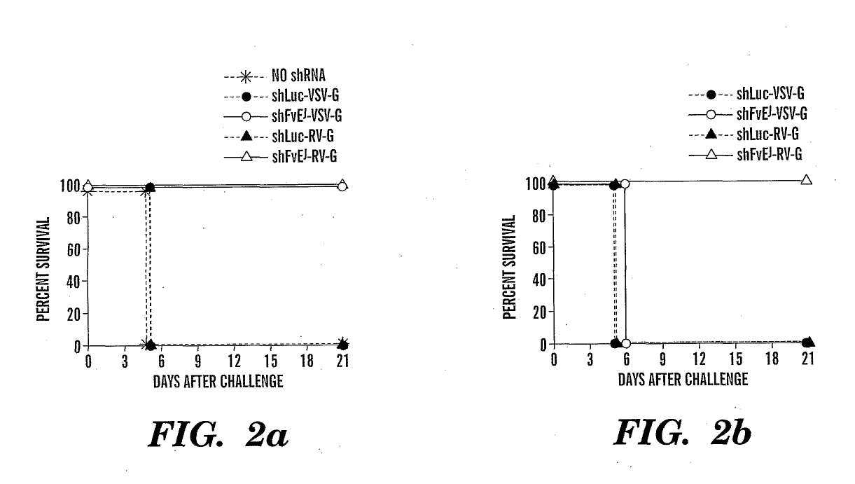

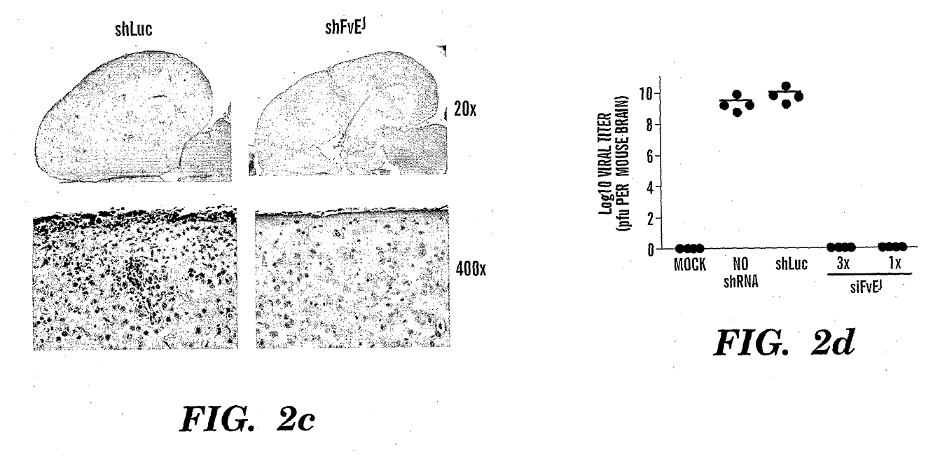

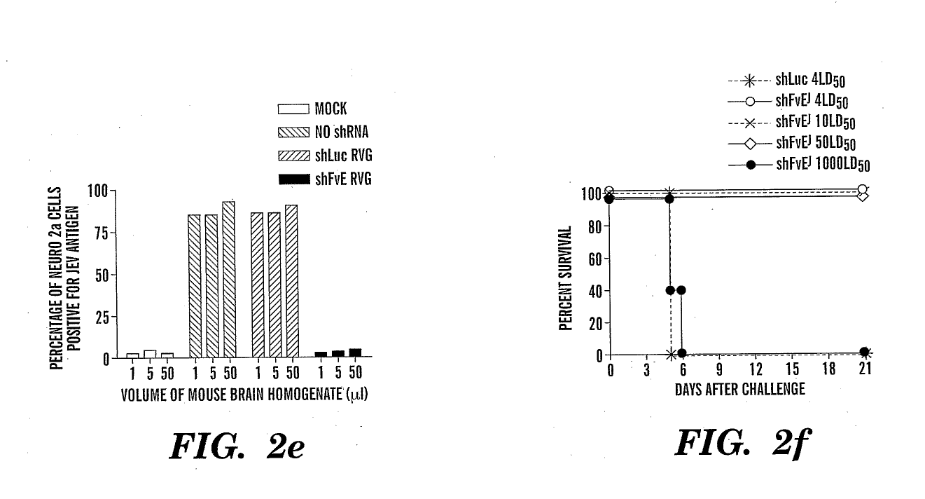

We next evaluated the potential of shFvEJ to protect against a lethal intracranial (ic) challenge with JEV. Balb/c mice were injected ic with the control shLuc or shFvEJ, pseudotyped with either VSV-G or RV-G. All mice were challenged with 4 LD50 of JEV injected at the same site and observed for mortality for 21 days. In the initial experiment (FIG. 2a) the mice received three ic injections with 2×105 transduction units (TU) of lentiviruses (the first at 4 days, the second at 2 days and the third 30 minutes before JEV challenge). JEV challenge in the control mock- or shLuc-injected mice induced the typical symptoms of viral encephalitis including ruffling of fur, hunching and hind limb weakness beginning on day 4 after infection, which rapidly progressed to paralysis, marked ataxia and death by 5 days (FIG. 2a, dashed lines). In contrast, none of the shFvEJ-injected mice (whether pseudotyped with VSV-G or RV-G) died or developed any of the clinical symptoms during the entire 21-day period of observation (FIG. 2a, solid lines). We also tested if a lower dose (2×103 TU) of lentivirus also conferred protection in a similar challenge experiment. Interestingly at this dose, all mice receiving VSV-G-pseudotyped shFvEJ succumbed by day 7 after viral challenge, whereas mice receiving RV-G-pseudotyped shFvEJ were completely protected (FIG. 2b). This enhanced protective efficacy is probably due to the capacity of RV-G pseudotyped lentivirus for retrograde axonal transport and increased lateral spread from the injection site18, resulting in a more extensive protection of neighboring cells. Brain sections from animals challenged with JEV were examined for pathological changes 5 days after infection. Brains of shLuc-treated mice showed the typical histopathological features of a diffuse, disseminated viral encephalitis with hemorrhage, extensive perivascular leukocyte infiltration and neuronal apoptosis, while no brain inflammation or neuropathology was observed in the shFvEJ-treated mice (FIG. 2c). Viral titration of brain homogenates revealed extremely high levels of viral replication in the control mice, whereas the shFvEJ-treated mice remained virus free (FIG. 2d). We also confirmed the lack of infectious virus by flow cytometry after an extended 5-day culture of Neuro 2a cells inoculated with the brain homogenates (FIG. 2e). Additionally, we confirmed that shFvEJ does not induce an IFN response by testing for expression of IFN response genes by RT-PCR using RNA from the lentivirus injected brains (FIG. 5) as well as by testing for serum type I IFN protein levels by ELISA (data not shown).

The previous set of experiments showed that 3 injections of a low dose of RV-G-pseudotyped shFvEJ can protect against a fatal JEV challenge. However, since 3 injections may not be necessary, we also tested if just one injection of RV-G-pseudotyped shFvEJ along with viral challenge could be equally effective. While injection with control shLuc did not modify the course of infection, even a single injection of shFvEJ was sufficient to protect mice completely against challenge with 4 LD50 of JEV (FIG. 2f). We also tested the ability of a single injection of shFvEJ to protect against increasing doses of challenge virus. Remarkably, a single injection with shFvEJ was able to afford complete protection with no detectable viral titers in the brain homogenates (FIG. 2e) even after challenge with 50 LD50 of JEV, although no protection was seen with the highest dose of 1000 LD50 (FIG. 2f). Collectively these results suggest that shFvEJ can confer a robust RNAi-mediated resistance to fatal Japanese encephalitis. Although the shRNA was co-administered with the challenge virus in these experiments, considering the lag time for the lentivirally transduced vector to be integrated in the host genome and processed into siRNA, the RNAi-mediated antiviral effect is likely to have been activated after JEV replication had already been initiated, suggesting that RNAi may be effective even when administered post infection.

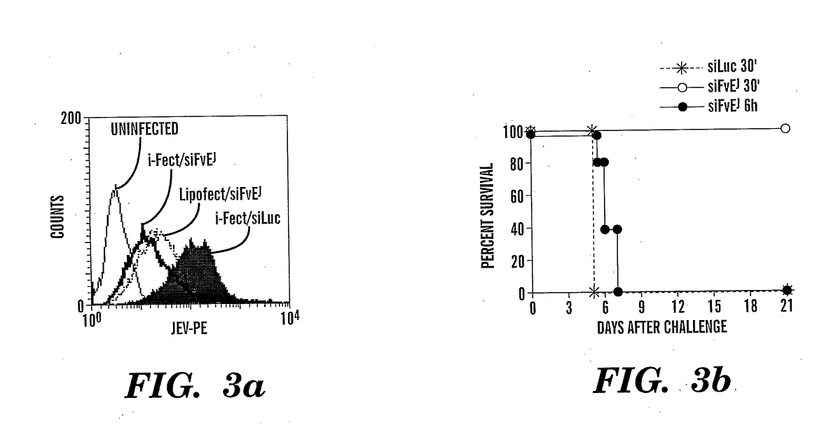

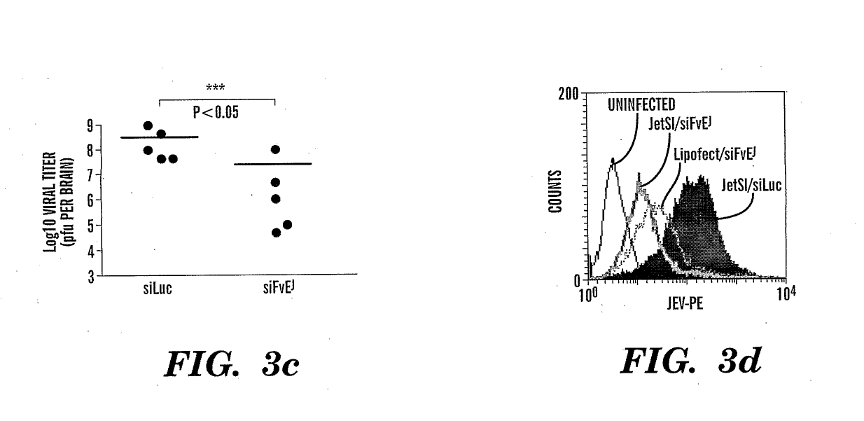

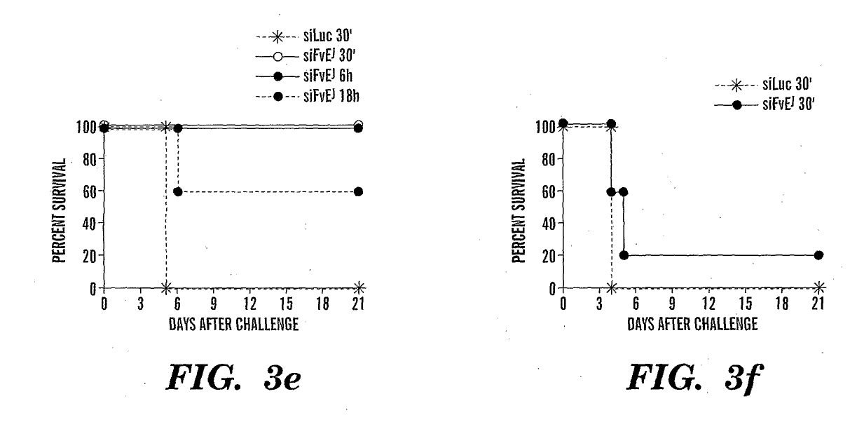

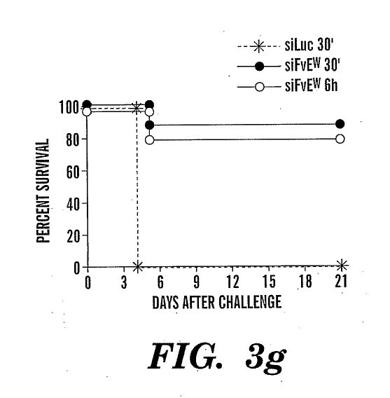

While our results so far showed that lentiviral delivery of shRNA can confer antiviral protection, this approach may not be ideal to treat humans because the long term effects of lentiviral integration is hard to predict. Moreover, the quantity of siRNA produced endogenously may be limiting for lentiviral delivery to be useful in a clinical setting as the brain cells are likely to contain high levels of viral RNA. On the other hand, similar to drug treatment, synthetic siRNA offers the possibility of escalating the dose for optimal viral suppression and is also potentially safer because of the transient nature of gene silencing. Thus, we also tested if FvEJ siRNA (siFvEJ) can protect mice against viral encephalitis. The cationic lipid formulation, I-FECT™ (Neuromics, Inc, MN) has been used for in vivo neuronal siRNA delivery without toxicity. After confirming that siFvEJ complexed with I-FECT™™ can silence JEV in Neuro 2a cells (FIG. 3A), we infected mice by ic injection with JEV and after allowing 30 min for viral adsorption, injected the synthetic siFvEJ or control luciferase siRNA (siLuc), complexed with I-FECT™ at the same site. All mice injected with siLuc died by day 5, whereas all of the siFvEJ injected mice survived indefinitely (FIG. 3B), suggesting that protection conferred by synthetic siRNA is similar to that by lentivirally delivered shRNA. We also tested if siRNA treatment can protect against an established JEV infection. Mice were first injected with JEV and siRNA complexed with I-FECT™ was injected 6 h later, a time point at which the viral RNA is being actively synthesized in the infected cells 19. Under these conditions, although siFvEJ was not able to prevent, it was able to delay death by 2-3 days (FIG. 3B). Moreover, mice treated 6 h post infection had brain viral titers 2 logs less than control mice, when tested on day 3 post infection (FIG. 3c). It should be pointed out that the available I-FECT™ formulation only allowed us to inject a total of approximately 6 μg (0.5 nmoles) of siRNA in the volume small enough to be safely injected by the ic route. Thus, it is possible that the limited amount of siRNA may not have been enough to spread sufficiently to protect cells away from the site of infection. If this were true, injection of a higher dose of siRNA should protect at later time points. To test this hypothesis, we used another combination cationic lipid formulation, JetSI and the fusogenic lipid dioleoylphosphatidylethanolamine (DOPE), which has also been used to deliver siRNA to brain cells in vivo without toxicity20. This formulation allowed us to inject higher amounts of siRNA in a small volume. After ascertaining that JetSI/DOPE can successfully deliver siFvEJ into Neuro 2a cells to inhibit JEV replication (FIG. 3d), we injected approximately 40 μg (3.2 nmoles) of siRNAs, complexed with JetSI/DOPE 30 min or 6 h after infection. While in both groups mice injected with the siLuc died within 5 days, siFvEJ-treated mice in both groups survived indefinitely (FIG. 3e). As with I-FECT™/siFvEJ-treated mice, neither IFN responsive genes nor IFN levels were increased after JetSI/DOPE/siFvEJ treatment (FIG. 5). Moreover, the siFvEJ-treated mice were completely healthy and brain sections taken 21 days after challenge showed no histopathological alterations, suggesting that the treatment was non-toxic. These results suggest that a single treatment with siFvEJ can protect against fatal encephalitis even when administered after the infection has already been established.

Next we tested if siRNA targeting the viral envelope gene can also suppress WNV. However, upon analysis, the B956 strain of WNV, used in the study was found to contain 6 nucleotide mismatches compared to the FvEJ sequence chosen from JEV. In fact, we found that lentivirally administered shFvEJ offers little protection from WNV encephalitis (FIG. 3f). It is worth mentioning that the inability of shFvEJ to protect against a mismatched WNV target reinforces our data that the siRNA protects from JEV infection by RNAi rather than by non-specific induction of IFN. To test if a fully matched siRNA protects against WNV, we designed a siRNA targeting the region corresponding to FvEJ, but with nucleotides matched completely with the WNV B956 sequence (FvEW). This sequence is also highly conserved in all the sequenced strains of WNV. After confirming that FvEW siRNA (siFvEW) inhibits WNV replication effectively in vitro (data not shown), we used the siRNA for in vivo studies. Control siLuc or siFvEW, complexed with JetSI/DOPE was injected at 30 minutes or 6 h after infection with WNV and the mice were observed for mortality. While all the mice injected with siLuc succumbed by d 5, 9/10 mice injected with siFvEW 30 min after WNV challenge and 4/5 mice receiving siFvEW 6 h after WNV challenge survived indefinitely (FIG. 3g). These results suggest that similar to siFvEJ treatment for JEV, siFvEW can protect against WNV encephalitis.

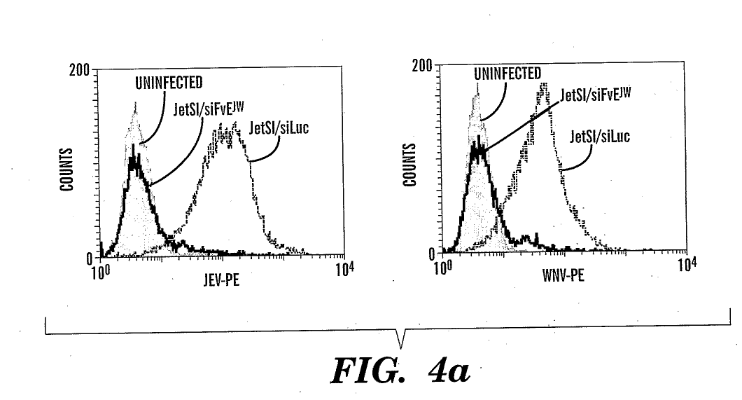

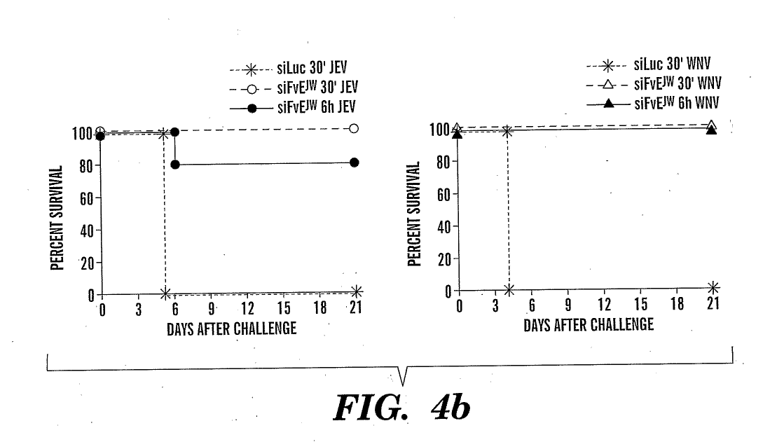

Encouraged by these results, we reasoned that it should be possible to design a common siRNA that can suppress both JEV and WNV. The flaviviral envelope glycoprotein is important in host cell receptor binding as well as in the internalization of the viral genome by membrane fusion. Probably because the fusion event is common to all flaviviruses, the cd loop in domain II of the E protein (aa 98-113), which is the region involved in fusion, is highly conserved among all flaviviruses at the amino acid level1. Although the FvEJ sequence is also derived from within this region (E protein aa 98-103), it is not completely conserved at the nucleotide level and as mentioned earlier, compared to JEV, the WNV strain that we used has multiple nucleotide changes. However, another region in the d loop (E protein, aa 105-111) is highly conserved between JEV, WNV as well as St. Louis encephalitis (SLE) virus even at the nucleotide level. Thus we designed a 21 nt siRNA (FvEJW), which is identical in sequence between the two viruses except for positions 3 and 21 in JEV and WNV target sequence respectively, where mutations are reported to be well tolerated with no significant effect on siRNA efficacy21. This siRNA was first tested for its ability to suppress both JEV and WNV in Neuro 2a cell line. In these cells, siFvEJW was found to be as effective as siFvEJ or siFvEW respectively in suppressing the replication of JEV as well as WNV (FIG. 4a). We also evaluated the ability of siFvEJW to cross-protect against a lethal challenge with JEV and WNV. Mice were first challenged with either JEV or WNV and after 30 min or 6 h injected ic with 3.2 nmoles of siFvEJW complexed with JetSI/DOPE. All mice injected with the control siLuc whether challenged with JE or WNV died. In contrast, all mice injected with siFvEJW 30 min after infection with either JE or WNV survived indefinitely. When siFvEJW was injected 6 h after challenge, 100% of mice challenged with WNV and 80% of mice challenged with JEV survived. Again, the specificity of the protective effects of FvEJW was verified by testing serum and brain homogenates of siRNA treated mice for nonspecific IFN induction as before (data not shown). Collectively these results indicate that the conserved siFvEJW can confer protection against both JE and WNV-induced encephalitis even when administered post infection. siFvEJW is likely to be effective in treating St. Louis encephalitis (SLE) as well because the target sequence is also very well conserved in all strains of SLE virus. Also, combinations of different siRNAs can be administered simultaneously.

Taken together, our study shows the considerable therapeutic potential of RNAi for treating viral encephalitis. Our results also shows that by careful design of conserved target sites, it is possible to use a single siRNA to suppress related viruses across species. This will be particularly important in treating acute and fatal viral infections, where the clinical symptoms often overlap and time does not permit exact etiologic diagnosis. Although our study suggests that a single lipid-based siRNA delivery in the brain parenchyma results in lateral spread and offers protection even in an established infection, this approach is unlikely to work when the infection has spread extensively to involve the entire brain. Thus, it is important to develop improved delivery methods. One approach is to use continuous intrathecal or intraventricular infusion with lipids and/or targeting with brain receptor-specific antibodies. In fact, these methods have been successfully used in other circumstances20,22. Moreover, pegylated immunoliposomes coated with transferrin receptor antibody has been successfully used for brain delivery of shRNA via the intravenous route11,23. With any of these methods even if some degree of reduction in viral load is achieved early in infection, the attenuation would increase the window period available for an immune response to develop that can eventually clear the infection. Although viral mutations even at the conserved sequence is a possibility, given the short time course of viral encephalitis, this is unlikely to be a major limiting factor. In summary, our study provides for translation of the relatively new RNAi technology from a laboratory tool into a viable clinical strategy for treating acute and deadly viral infections.

### Example 2

Flaviviral genomes share a basic genomic structure. We selected several genes for siRNA targeting (FIG. 6A). The sense and antisense strands of the siRNAs were as follows and their genomic target sequences are presented in FIG. 6A:

| | | |
|---|---|---|
| FvC | | |
| target: | CTATCAATATGCTGAACGCG | (SEQ ID NO: 96) |
| sense: | CUAUCAAUAUGCUGAACGCG | (SEQ ID NO: 1) |
| antisense: | CGCGUUCAGCAUAUUGAUAG | (SEQ ID NO: 2) |
| FvE | | |
| target: | CGGATGTGGACTTTTCGGG | (SEQ ID NO: 97) |
| sense: | CGGAUGUGGACUUUUCGGG | (SEQ ID NO: 3) |
| antisense: | CCCGAAAAGUCCACAUCCG | (SEQ ID NO: 4) |
| FvNS3 | | |
| target: | GACAGAAGGTGGTGTTTGAT | (SEQ ID NO: 98) |
| sense: | GACAGAAGGUGGUGUUUGAU | (SEQ ID NO: 5) |
| antisense: | AUCAAACACCACCUUCUGUC | (SEQ ID NO: 6) |
| FvR1 | | |
| target: | CAGCATATTGACACCTGGG | (SEQ ID NO: 99) |
| sense: | CAGCAUAUUGACACCUGGG | (SEQ ID NO: 7) |
| antisense: | CCCAGGUGUCAAUAUGCUG | (SEQ ID NO: 8) |
| FvR2 | | |
| target: | GGACTAGAGGTTAGAGGAG | (SEQ ID NO: 100) |
| sense: | GGACUAGAGGUUAGAGGAG | (SEQ ID NO: 9) |
| antisense: | CUCCUCUAACCUCUAGUCC | (SEQ ID NO: 10) |
| DEN-E3 | | |
| target: | ACACAACATGGAACAATAG | (SEQ ID NO: 104) |
| sense: | ACACAACAUGGAACAAUAG | (SEQ ID NO: 32) |
| antisense: | CUAUUGUUCCAUGUUGUGU | (SEQ ID NO: 33) |
| DEN-E4 | | |
| target: | CATAGAAGCAGAACCTCCA | (SEQ ID NO: 105) |
| sense: | CAUAGAAGCAGAACCUCCA | (SEQ ID NO: 34) |
| antisense: | UGGAGGUUCUGCUUCUAUG | (SEQ ID NO: 35) |

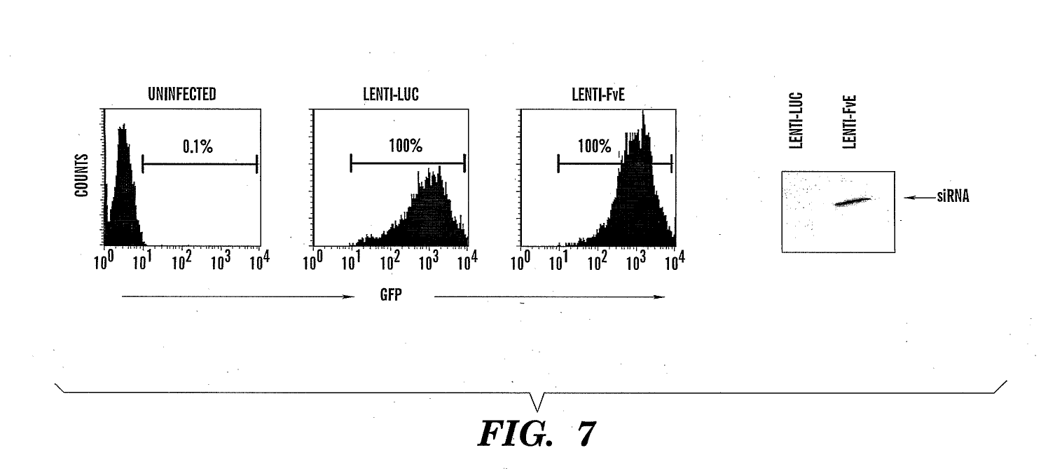

These siRNAs were incorporated into lentivirus constructs for transduction into target cells and virus replication was measured two days later (FIG. 6B). The siRNAs FvE, DN-E4, DN-E3, FvC and FvR1 significantly inhibited replication of Dengue virus as compared to the mock-transfected cells. Percentages of virus replication are shown in FIG. 6B. The Ig-control represents an antibody that effectively inhibits viral replication. The FvE siRNA construct was stably transduced in BHK21 cells and generated FvE siRNA expression as shown in the Northern Blot analysis (FIG. 7). Transfection efficiency was shown using a GFP gene as a reporter (FIG. 7) and was shown to be nearly 100%.

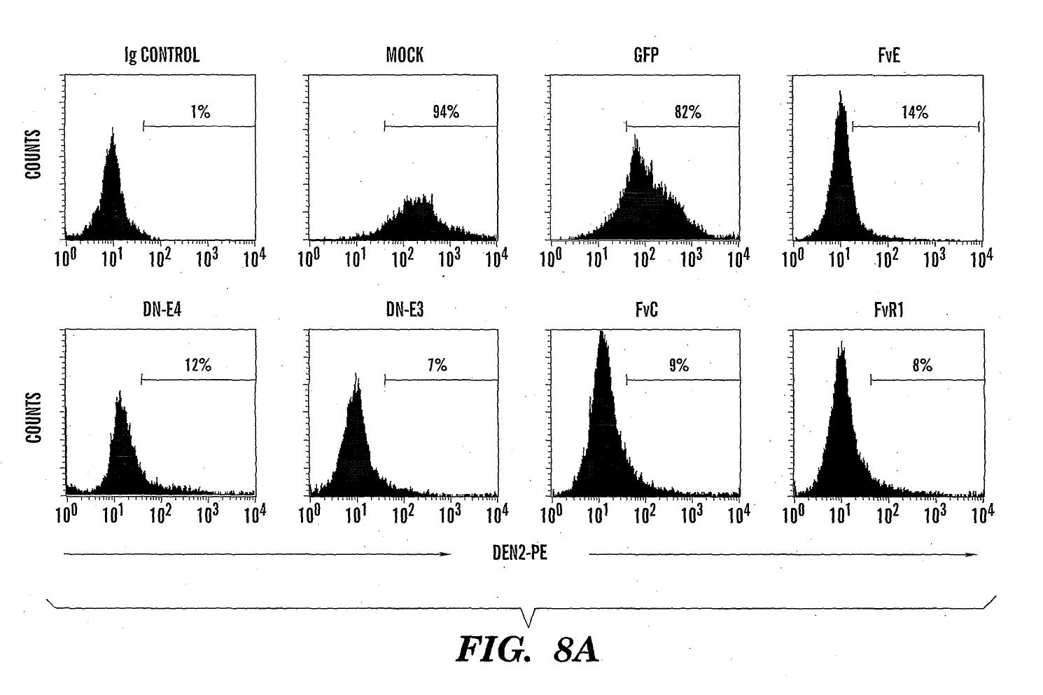

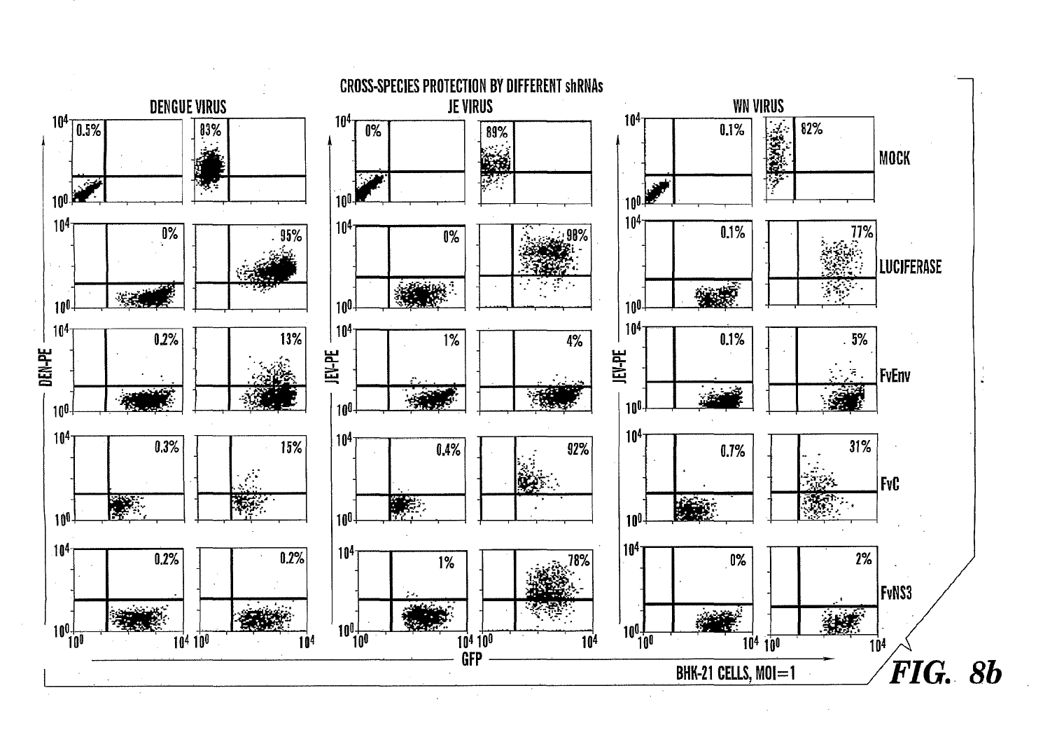

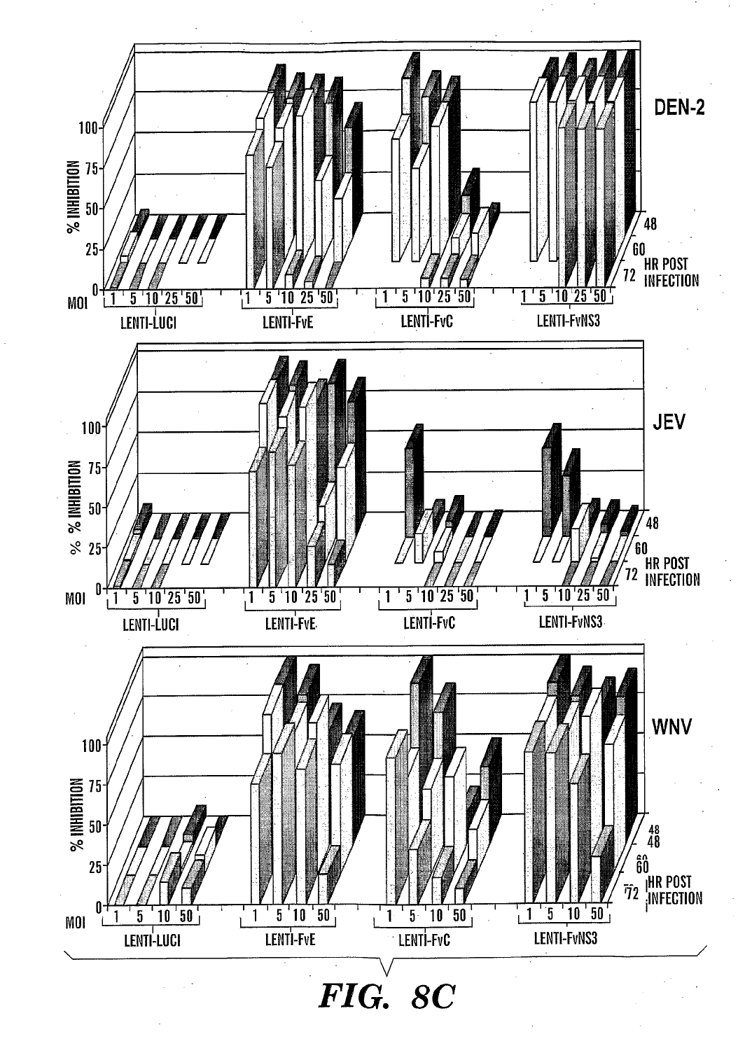

BHK21 cells were transfected with siRNAs targeting several genes and infected with flavivirus. Dengue virus replication was inhibited by all of the siRNAs transfected (FIG. 8A).

The relative protection provided by the FvE, FvC and FvNS3 siRNAs was compared between Dengue virus, JE virus and WN virus infections (FIG. 8B). The duration of the protection provided by the FvE, FvC and FvNS3 siRNAs at different MOI's was also compared between the Dengue, JE and WN viruses.

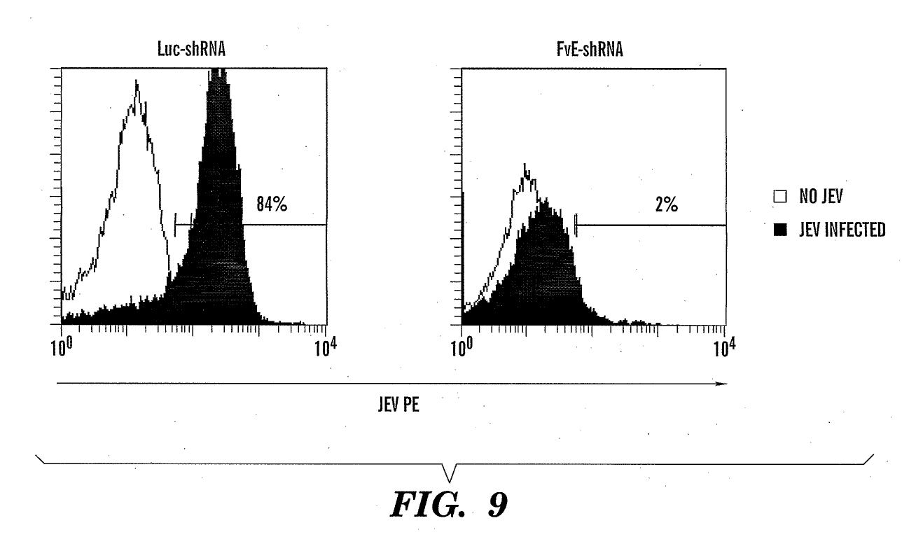

Mice treated with FvE-shRNA and Luc-shRNA (control) were infected with JE virus. The FvE shRNA treated mice showed an absence of virus in the brains (FIG. 9).

### Example 3

Additional target sequences for siRNAs to inhibit flaviviruses and to treat flavivirus infection were deduced based upon regions that are conserved between different flavivirus serotypes. Table 4 contains target sequences conserved between West Nile virus and Japanese encephalitis virus. Table 5 lists sequences that are conserved between different Dengue virus serotypes.

TABLE 4

Additional sequences conserved
between JE and WN viruses

| | |
|---|---|
| | Identical in >18 |
| Target | residues |

| | | | | |
|---|---|---|---|---|
| gene (nt | Target sequence | Seq | WNV | JEV |
| position) | (21 nt) | ID | (n = 31) | (n = 22) |

| | | | | |
|---|---|---|---|---|
| NS3 | | | | |
| 5009-5029 | GGAACATCAGGCTCACCAATA | 106 | 97% | 95% |
| 5054-5074 | GGGCTTTATGGCAATGGAGTC | 107 | 100% | 13% |
| | A | | | |
| 5223-5243 | TCTGCCACAGATCATCAAAGA | 108 | 97% | 95% |
| 5293-5313 | GTGGCTGCTGAGATGGCTGAA | 109 | 97% | 0% |
| 5407-5427 | CTCACCCACAGGCTGATGTCT | 110 | 97% | 0% |
| 5458-5478 | GTGATGGATGAGGCTCATTTC | 111 | 94% | 100% |
| 5654-5674 | GATACGAATGGATCACAGAAT | 112 | 94% | 100% |
| NS5 | | | | |
| 7974-7994 | GGAAGTCAGAGGGTACACAAA | 113 | 94% | 100% |
| 8049-8069 | GGTCACCATGAAGAGTGGAGT | 114 | 97% | 86% |
| 8321-8341 | ACTCCACGCACGAGATGTGTT | 115 | 97% | 95% |
| 8705-8725 | CCATGGCCATGACTGACACTA | 116 | 100% | 95% |
| 9103-9123 | GCCATTTGGTTCATGTGGCTT | 117 | 100% | 100% |
| 9625-9645 | TGGACCTGGCTGTTTGAGAAT | 118 | 100% | 95% |

TABLE 5

Sequences conserved among
the dengue virus serotypes

| | | | |
|---|---|---|---|
| | | | identical |
| | | Seq | in >18 |
| Gene | Target sequence | ID | residues |

| | | | | |
|---|---|---|---|---|
| Dengue | E | AATATCAAACACCACCACCGA | 119 | 91% |
| serotype | | AAAGCTTTGAAACTAAGCTGG | 120 | 92% |
| 1* | NS3 | AAGAAGGGCCTCTACCAGAGA | 121 | 56% |
| | | AAGGGATTATCCCAGCCCTCT | 122 | 70% |
| | NS5 | AAGAGGTGGCTGGTCATATTA | 123 | 65% |
| ²Dengue | E | AAATGAAGAGCAGGACAAAAG | 124 | 100% |
| serotype | | AAATTGGATACAGAAAGAGAC | 125 | 100% |
| 2* | | AAACACAACATGGAACAATAG | 126 | 100% |
| | | AACATAGAAGCAGAACCTCCA | 127 | 100% |
| | NS3 | AAAGGGAAGACTGTTTGGTTC | 128 | 75% |
| | | AAAAGGAAAAGTTGTGGGTCT | 129 | 75% |
| | NS5 | AATGGCCATCAGTGGAGATGA | 130 | 82% |
| | | AAAGGTGAGAAGCAATGCAGC | 131 | 80% |
| ³Dengue | E | AAAAGCAAGAAGTAGTTGTCC | 132 | 86% |
| serotype | | AAAATTGGAATAGGTGTCCTC | 133 | 87% |
| 3* | NS5 | AAAATCCTTACAAAACGTGGG | 134 | 88% |
| | | AAATCCTTACAAAACGTGGGC | 135 | 88 |

The references cited below and throughout the specification are incorporated herein in their entirety by reference.

### REFERENCES

1. Rey, F. A., Heinz, F. X., Mandl, C., Kunz, C. & Harrison, S. C. The envelope glycoprotein from tick-borne encephalitis virus at 2 A resolution. Nature 375, 291-8 (1995).
2. Biggerstaff, B. J. & Petersen, L. R. Estimated risk of transmission of the West Nile virus through blood transfusion in the US, 2002. Transfusion 43, 1007-17 (2003).
3. Tyler, K. L. West Nile virus infection in the United States. Arch Neurol 61, 1190-5 (2004).
4. Mackenzie, J. S., Gubler, D. J. & Petersen, L. R. Emerging flaviviruses: the spread and resurgence of Japanese encephalitis, West Nile and dengue viruses. Nat Med 10, S98-109 (2004).
5. Solomon, T. Flavivirus encephalitis. N Engl J Med 351, 370-8 (2004).
6. Seligman, S. J. & Gould, E. A. Live flavivirus vaccines: reasons for caution. Lancet 363, 2073-5 (2004).
7. Novina, C. D. et al. siRNA-directed inhibition of HIV-1 infection. Nat Med 8, 681-6 (2002).
8. Bennink, J. R. & Palmore, T. N. The promise of siRNAs for the treatment of influenza. Trends Mol Med 10, 571-4 (2004).
9. Bitko, V., Musiyenko, A., Shulyayeva, O. & Barik, S. Inhibition of respiratory viruses by nasally administered siRNA. Nat Med 11, 50-5 (2005).
10. Xia, H. et al. RNAi suppresses polyglutamine-induced neurodegeneration in a model of spinocerebellar ataxia. Nat Med 10, 816-20 (2004).
11. Zhang, Y., Boado, R. J. & Pardridge, W. M. In vivo knockdown of gene expression in brain cancer with intravenous RNAi in adult rats. J Gene Med 5, 1039-45 (2003).
12. Shankar, P., Manjunath, N. & Lieberman, J. The prospect of silencing disease using RNA interference. Jama 293, 1367-73 (2005).
13. Ralph, G. S. et al. Silencing mutant SOD1 using RNAi protects against neurodegeneration and extends survival in an ALS model. Nat Med 11, 429-33 (2005).
14. Monath, T. P. & Heinz, F. X. (eds. Fields, B. N., Knipe, M. & Howley, P. M.) (Lippincott-Raven, Philadelphia, Pa., 1996).
15. Chambers, T. J., Hahn, C. S., Galler, R. & Rice, C. M. Flavivirus genome organization, expression, and replication. Annu Rev Microbiol 44, 649-88 (1990).
16. Rubinson, D. A. et al. A lentivirus-based system to functionally silence genes in primary mammalian cells, stem cells and transgenic mice by RNA interference. Nat Genet 33, 401-6 (2003).
17. Mosca, J. D. & Pitha, P. M. Transcriptional and posttranscriptional regulation of exogenous human beta interferon gene in simian cells defective in interferon synthesis. Mol Cell Biol 6, 2279-83 (1986).
18. Mazarakis, N. D. et al. Rabies virus glycoprotein pseudotyping of lentiviral vectors enables retrograde axonal transport and access to the nervous system after peripheral delivery. Hum Mol Genet 10, 2109-21 (2001).
19. Uchil, P. D. & Satchidanandam, V. Characterization of RNA synthesis, replication mechanism, and in vitro RNA-dependent RNA polymerase activity of Japanese encephalitis virus. Virology 307, 358-71 (2003).
20. Hassani, Z. et al. Lipid-mediated siRNA delivery down-regulates exogenous gene expression in the mouse brain at picomolar levels. J Gene Med 7, 198-207 (2005).
21. Du, Q., Thonberg, H., Wang, J., Wahlestedt, C. & Liang, Z. A systematic analysis of the silencing effects of an active siRNA at all single-nucleotide mismatched target sites. Nucleic Acids Res 33, 1671-7 (2005).
22. Thakker, D. R. et al. Neurochemical and behavioral consequences of widespread gene knockdown in the adult mouse brain by using nonviral RNA interference. Proc Natl Acad Sci USA 101, 17270-5 (2004).
23. Zhang, Y. et al. Intravenous RNA interference gene therapy targeting the human epidermal growth factor receptor prolongs survival in intracranial brain cancer. Clin Cancer Res 10, 3667-77 (2004).
24. Virology: A Practical Approach (ed. Maly, B. W. J.) (IRL Press, Oxford, UK, 1985).
25. Stewart, S. A. et al. Lentivirus-delivered stable gene silencing by RNAi in primary cells. Rna 9, 493-501 (2003).
26. Song, E. et al. Sustained small interfering RNA-mediated human immunodeficiency virus type 1 inhibition in primary macrophages. J Virol 77, 7174-81 (2003).

We claim:

1. A method of inhibiting expression of flavivirus mRNA, or an alternative splice form, mutant or cognate thereof, or preventing or treating flavivirus mediated disease, comprising administering to a subject an effective amount of at least one isolated siRNA or shRNA comprising an RNA duplex comprised of one or two molecules, wherein a portion of the molecule comprises a nucleotide sequence identical to a target sequence of about 15 to about 30 contiguous nucleotides in flavivirus mRNA or mutant or variant thereof.

2. The method of claim 1, wherein the flavivirus mRNA is selected from the group consisting of capsid encoding gene, envelope encoding gene, non-structural protein 3 encoding gene, untranslated regions and any combination thereof.

3. The method of claim 1, wherein the at least one siRNA is selected from the group consisting of SEQ ID NO: 1 and SEQ ID NO: 2; SEQ ID NO: 3 and SEQ ID NO: 4; SEQ ID NO: 5 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16 and any combination thereof.

4. The method of claim 19, wherein the vertebrate is a human.

5. The method of claim 1, wherein expression of flavivirus mRNA, or an alternative splice form, mutant or cognate thereof is inhibited in the brain, cerebral-spinal tissue, body tissue of the subject.

6. The method of claim 1, wherein the effective amount of the siRNA is from about 1 nM to about 100 nM.

7. The method of claim 1, wherein the siRNA is administered in conjunction with a delivery reagent.

8. The method of claim 7, wherein the delivery agent is selected from the group consisting of lipofection agents.

9. The method of claim 7, wherein the delivery agent is a liposome.

10. The method of claim 9, wherein the liposome comprises a ligand which targets the liposome to cells at or near the site of infection.

11. The method of claim 10, wherein the ligand binds to receptors on the brain endothelial cells.

12. The method of claim 10, wherein the ligand comprises a monoclonal antibody.

13. The method of claim 10, wherein the liposome is modified with an opsonization-inhibition moiety.

14. The method of claim 13, wherein the an opsonization-inhibition moiety comprises a PEG, PPG, or derivative thereof.

15. The method of claim 1, wherein the siRNA is expressed from an vector.

16. The method of claim 1, wherein the siRNA is administered by an enteral administration route.

17. The method of claim 1, wherein the enteral administration route is selected from the group consisting of oral, rectal, and intranasal.

18. The method of claim 1, wherein the siRNA is administered by a parenteral administration route.

19. The method of claim 18, wherein the parenteral administration route is selected from the group consisting of intravascular administration, peri- and intra-tissue administration, subcutaneous injection or deposition, subcutaneous infusion, intraocular administration, and direct application.

20. The method of claim 19, wherein the intravascular administration is selected from the group consisting of intravenous bolus injection, intravenous infusion, intra-arterial bolus injection, intra-arterial infusion and catheter instillation into the vasculature.

21. The method of claim 20, wherein the direct application comprises application by catheter, corneal pellet, eye dropper, suppository, an implant comprising a porous material, an implant comprising a non-porous material, or an implant comprising a gelatinous material.

22. The method of claim 21, wherein the implant is biodegradable.

23. The method of claim 1, wherein the siRNA is administered in combination with a pharmaceutical agent for treating, alleviating symptoms relating to, and/or preventing infection secondary to flavivirus disease, which pharmaceutical agent is different from the siRNA and is selected from the group consisting of anticonvulsants, antinausea medicants, antibiotics for prevention of pneumonia and/or urinary tract infection or any combination thereof.

24. A method of treating flavivirus infection comprising administering to a subject infected or suspected to have been infected with a flavivirus an siRNA or shRNA comprising an RNA duplex comprised of one or two molecules, wherein a portion of the molecule comprises a nucleotide sequence identical to a target sequence of about 15 to about 30 contiguous nucleotides in flavivirus mRNA or mutant or variant thereof and a pharmaceutical carrier, wherein said siRNA or shRNA binds the target sequence and results in inhibition of viral protein production thereby treating the flavivirus infection.

25. A method of preventing flavivirus infection comprising administering to a subject an siRNA or shRNA comprising an RNA duplex comprised of one or two molecules, wherein a portion of the molecule comprises a nucleotide sequence identical to a target sequence of about 15 to about 30 contiguous nucleotides in flavivirus mRNA or mutant or variant thereof and a pharmaceutical carrier, wherein said siRNA or shRNA binds the target sequence and results in inhibition of viral protein production thereby preventing the flavivirus infection.

26. The method of claim 25, wherein the administering is performed in daily intervals during the time the individual is susceptible for a flavivirus infection.

27. The method of claim 25, wherein the administering is performed in weekly intervals during the time the individual is susceptible for a flavivirus infection.

28. The method of claim 25, wherein the administering is performed in monthly intervals during the time the individual is susceptible for a flavivirus infection.

29. An isolated siRNA or shRNA comprising an RNA duplex comprised of one or two molecules, wherein a portion of the molecule comprises a nucleotide sequence identical to a target sequence of about 15 to about 30 contiguous nucleotides in flavivirus mRNA or mutant or variant thereof.

30. The siRNA of claim 29, wherein the flavivirus is selected from the group consisting of Cacipacore virus, Koutango virus, Murray Valley encephalitis virus, St. Louis Encephalitis virus, Alfuy virus, Kunjin virus, Yaounde virus, West Nile virus, Japanese Encephalitis virus, Dengue virus or any combination thereof.

31. The siRNA of claim 29, wherein the flavivirus is selected from the group consisting of West Nile virus, Japanese Encephalitis virus, Dengue virus or any combination thereof.

32. The siRNA of claim 29, wherein the target sequence is conserved between at least 2 flaviviruses.

33. The siRNA of claim 29, wherein the target is a is selected from a group consisting of capsid encoding gene, envelope encoding gene, non-structural protein 3 encoding gene, untranslated regions and any combination thereof.

34. The siRNA of claim 29, wherein the sense RNA strand comprises SEQ ID NO: 1, and the antisense strand comprises SEQ ID NO: 2.

35. The siRNA of claim 29, wherein the sense RNA strand comprises SEQ ID NO: 3, and the antisense strand comprises SEQ ID NO: 4.

36. The siRNA of claim 29, wherein the sense RNA strand comprises SEQ ID NO: 5, and the antisense strand comprises SEQ ID NO: 6.

37. The siRNA of claim 29, wherein the sense RNA strand comprises SEQ ID NO: 7, and the antisense strand comprises SEQ ID NO: 8.

38. The siRNA of claim 29, wherein the sense RNA strand comprises SEQ ID NO: 9, and the antisense strand comprises SEQ ID NO: 10.

39. The siRNA of claim 29, wherein the sense RNA strand comprises SEQ ID NO: 11, and the antisense strand comprises SEQ ID NO: 12.

40. The siRNA of claim 29, wherein the sense RNA strand comprises SEQ ID NO: 13, and the antisense strand comprises SEQ ID NO: 14.

41. The siRNA of claim 29, wherein the sense RNA strand comprises SEQ ID NO: 15, and the antisense strand comprises SEQ ID NO: 16.

42. A pharmaceutical composition comprising an isolated siRNA of claim 1 and a pharmaceutically acceptable carrier.

43. The pharmaceutical composition of claim 42, further comprising lipofection agents.


SEQUENCE LISTING (138 sequences)

SEQ ID NO: 1 - RNA, 20 nt, Artificial Sequence
  Description of Artificial Sequence Synthetic oligonucleotide
cuaucaauaugcugaacgcg

SEQ ID NO: 2 - RNA, 20 nt, Artificial Sequence
  Description of Artificial Sequence Synthetic oligonucleotide
cgcguucagcauauugauag

SEQ ID NO: 3 - RNA, 19 nt, Artificial Sequence
  Description of Artificial Sequence Synthetic oligonucleotide
cggauguggacuuuucggg

SEQ ID NO: 4 - RNA, 19 nt, Artificial Sequence
  Description of Artificial Sequence Synthetic oligonucleotide
cccgaaaaguccacauccg

SEQ ID NO: 5 - RNA, 20 nt, Artificial Sequence
  Description of Artificial Sequence Synthetic oligonucleotide
gacagaaggugguguuugau

SEQ ID NO: 6 - RNA, 20 nt, Artificial Sequence
  Description of Artificial Sequence Synthetic oligonucleotide
aucaaacaccaccuucuguc

SEQ ID NO: 7 - RNA, 19 nt, Artificial Sequence
  Description of Artificial Sequence Synthetic oligonucleotide
cagcauauugacaccuggg

SEQ ID NO: 8 - RNA, 19 nt, Artificial Sequence
  Description of Artificial Sequence Synthetic oligonucleotide
cccaggugucaauaugcug

SEQ ID NO: 9 - RNA, 19 nt, Artificial Sequence
  Description of Artificial Sequence Synthetic oligonucleotide
ggacuagagguuagaggag

SEQ ID NO: 10 - RNA, 19 nt, Artificial Sequence
  Description of Artificial Sequence Synthetic oligonucleotide
cuccucuaaccucuagucc

SEQ ID NO: 11 - RNA, 19 nt, Artificial Sequence
  Description of Artificial Sequence Synthetic oligonucleotide
ggauguggacuuuucggga

SEQ ID NO: 12 - RNA, 19 nt, Artificial Sequence
  Description of Artificial Sequence Synthetic oligonucleotide
ucccgaaaaguccacaucc

SEQ ID NO: 13 - RNA, 19 nt, Artificial Sequence
  Description of Artificial Sequence Synthetic oligonucleotide
ggcugcggacuguuuggaa

SEQ ID NO: 14 - RNA, 19 nt, Artificial Sequence
  Description of Artificial Sequence Synthetic oligonucleotide
uuccaaacaguccgcagcc

SEQ ID NO: 15 - RNA, 21 nt, Artificial Sequence
  Description of Artificial Sequence Synthetic oligonucleotide
gggagcauugacacaugugca

SEQ ID NO: 16 - RNA, 21 nt, Artificial Sequence
  Description of Artificial Sequence Synthetic oligonucleotide
ugcacaugugucaaugcuccc

SEQ ID NO: 17 - protein, 9 aa, Human immunodeficiency virus
RKKRRQRRR

SEQ ID NO: 18 - protein, 13 aa, Human immunodeficiency virus
GRKKRRQRRRTPQ

SEQ ID NO: 19 - protein, 11 aa, Human immunodeficiency virus
YGRKKRRQRRR

SEQ ID NO: 20 - protein, 16 aa, Artificial Sequence
  Description of Artificial Sequence Synthetic Kaposi fibroblast growth factor peptide
AAVALLPAVLLALLAP

SEQ ID NO: 21 - protein, 17 aa, Caiman crocodylus
MGLGLHLLVLAAALQGA

SEQ ID NO: 22 - protein, 17 aa, Human immunodeficiency virus
GALFLGFLGAAGSTMGA

SEQ ID NO: 23 - protein, 16 aa, Drosophila sp.
RQIKIWFQNRRMKWKK

SEQ ID NO: 24 - protein, 5 aa, Artificial Sequence
  Description of Artificial Sequence Synthetic peptide
XRGDX

SEQ ID NO: 25 - protein, 24 aa, Influenza virus
GLFEAIAGFIENGWEGMIDGGGYC

SEQ ID NO: 26 - protein, 27 aa, Artificial Sequence
  Description of Artificial Sequence Synthetic transportan A peptide
GWTLNSAGYLLGKINLKALAALAKKIL

SEQ ID NO: 27 - protein, 9 aa, Artificial Sequence
  Description of Artificial Sequence Synthetic Pre-S-peptide
SDHQLNPAF

SEQ ID NO: 28 - protein, 9 aa, Artificial Sequence
  Description of Artificial Sequence Synthetic Somatostatin peptide
SFCYWKTCT

SEQ ID NO: 29 - DNA, 19 bp, Artificial Sequence
  Description of Artificial Sequence Synthetic oligonucleotide
ggatgtggacttttcggga

SEQ ID NO: 30 - DNA, 19 bp, Artificial Sequence
  Description of Artificial Sequence Synthetic oligonucleotide
ggatgtggactatttggaa

SEQ ID NO: 31 - DNA, 19 bp, Artificial Sequence
  Description of Artificial Sequence Synthetic oligonucleotide
ggctgcggactgtcgggga

SEQ ID NO: 32 - RNA, 19 nt, Artificial Sequence
  Description of Artificial Sequence Synthetic oligonucleotide
acacaacauggaacaauag

SEQ ID NO: 33 - RNA, 19 nt, Artificial Sequence
  Description of Artificial Sequence Synthetic oligonucleotide
cuauuguuccauguugugu

SEQ ID NO: 34 - RNA, 19 nt, Artificial Sequence
  Description of Artificial Sequence Synthetic oligonucleotide
cauagaagcagaaccucca

SEQ ID NO: 35 - RNA, 19 nt, Artificial Sequence
  Description of Artificial Sequence Synthetic oligonucleotide
uggagguucugcuucuaug

SEQ ID NO: 36 - RNA, 21 nt, Artificial Sequence
  Description of Artificial Sequence Synthetic oligonucleotide
ggaacaucaggcucaccaaua

SEQ ID NO: 37 - RNA, 21 nt, Artificial Sequence
  Description of Artificial Sequence Synthetic oligonucleotide
uauuggugagccugauguucc

SEQ ID NO: 38 - RNA, 22 nt, Artificial Sequence
  Description of Artificial Sequence Synthetic oligonucleotide
gggcuuuauggcaauggaguca

SEQ ID NO: 39 - RNA, 22 nt, Artificial Sequence
  Description of Artificial Sequence Synthetic oligonucleotide
ugacuccauugccauaaagccc

SEQ ID NO: 40 - RNA, 21 nt, Artificial Sequence
  Description of Artificial Sequence Synthetic oligonucleotide
ucugccacagaucaucaaaga

SEQ ID NO: 41 - RNA, 21 nt, Artificial Sequence
  Description of Artificial Sequence Synthetic oligonucleotide
ucuuugaugaucuguggcaga

SEQ ID NO: 42 - RNA, 21 nt, Artificial Sequence
  Description of Artificial Sequence Synthetic oligonucleotide
guggcugcugagauggcugaa

SEQ ID NO: 43 - RNA, 21 nt, Artificial Sequence
  Description of Artificial Sequence Synthetic oligonucleotide
uucagccaucucagcagccac

SEQ ID NO: 44 - RNA, 21 nt, Artificial Sequence
  Description of Artificial Sequence Synthetic oligonucleotide
cucacccacaggcugaugucu

SEQ ID NO: 45 - RNA, 21 nt, Artificial Sequence
  Description of Artificial Sequence Synthetic oligonucleotide
agacaucagccugugggugag

SEQ ID NO: 46 - RNA, 21 nt, Artificial Sequence
  Description of Artificial Sequence Synthetic oligonucleotide
gugauggaugaggcucauuuc

SEQ ID NO: 47 - RNA, 21 nt, Artificial Sequence
  Description of Artificial Sequence Synthetic oligonucleotide
gaaaugagccucauccaucac

SEQ ID NO: 48 - RNA, 21 nt, Artificial Sequence
  Description of Artificial Sequence Synthetic oligonucleotide
gauacgaauggaucacagaau

SEQ ID NO: 49 - RNA, 21 nt, Artificial Sequence
  Description of Artificial Sequence Synthetic oligonucleotide
auucugugauccauucguauc

SEQ ID NO: 50 - RNA, 21 nt, Artificial Sequence
  Description of Artificial Sequence Synthetic oligonucleotide
ggaagucagaggguacacaaa

SEQ ID NO: 51 - RNA, 21 nt, Artificial Sequence
  Description of Artificial Sequence Synthetic oligonucleotide
uuuguguacccucugacuucc

SEQ ID NO: 52 - RNA, 21 nt, Artificial Sequence
  Description of Artificial Sequence Synthetic oligonucleotide
ggucaccaugaagaguggagu

SEQ ID NO: 53 - RNA, 21 nt, Artificial Sequence
  Description of Artificial Sequence Synthetic oligonucleotide
acuccacucuucauggugacc

SEQ ID NO: 54 - RNA, 21 nt, Artificial Sequence
  Description of Artificial Sequence Synthetic oligonucleotide
acuccacgcacgagauguguu

SEQ ID NO: 55 - RNA, 21 nt, Artificial Sequence
  Description of Artificial Sequence Synthetic oligonucleotide
aacacaucucgugcguggagu

SEQ ID NO: 56 - RNA, 21 nt, Artificial Sequence
  Description of Artificial Sequence Synthetic oligonucleotide
ccauggccaugacugacacua

SEQ ID NO: 57 - RNA, 21 nt, Artificial Sequence
  Description of Artificial Sequence Synthetic oligonucleotide
uagugucagucauggccaugg

SEQ ID NO: 58 - RNA, 21 nt, Artificial Sequence
  Description of Artificial Sequence Synthetic oligonucleotide
gccauuugguucauguggcuu

SEQ ID NO: 59 - RNA, 21 nt, Artificial Sequence
  Description of Artificial Sequence Synthetic oligonucleotide
aagccacaugaaccaaauggc

SEQ ID NO: 60 - RNA, 21 nt, Artificial Sequence
  Description of Artificial Sequence Synthetic oligonucleotide
uggaccuggcuguuugagaau

SEQ ID NO: 61 - RNA, 21 nt, Artificial Sequence
  Description of Artificial Sequence Synthetic oligonucleotide
auucucaaacagccaggucca

SEQ ID NO: 62 - RNA, 21 nt, Artificial Sequence
  Description of Artificial Sequence Synthetic oligonucleotide
aauaucaaacaccaccaccga

SEQ ID NO: 63 - RNA, 21 nt, Artificial Sequence
  Description of Artificial Sequence Synthetic oligonucleotide
ucgguggugguguuugauauu

SEQ ID NO: 64 - RNA, 21 nt, Artificial Sequence
  Description of Artificial Sequence Synthetic oligonucleotide
aaagcuuugaaacuaagcugg

SEQ ID NO: 65 - RNA, 21 nt, Artificial Sequence
  Description of Artificial Sequence Synthetic oligonucleotide
ccagcuuaguuucaaagcuuu

SEQ ID NO: 66 - RNA, 21 nt, Artificial Sequence
  Description of Artificial Sequence Synthetic oligonucleotide
aagaagggccucuaccagaga

SEQ ID NO: 67 - RNA, 21 nt, Artificial Sequence
  Description of Artificial Sequence Synthetic oligonucleotide
ucucugguagaggcccuucuu

SEQ ID NO: 68 - RNA, 21 nt, Artificial Sequence
  Description of Artificial Sequence Synthetic oligonucleotide
aagggauuaucccagcccucu

SEQ ID NO: 69 - RNA, 21 nt, Artificial Sequence
  Description of Artificial Sequence Synthetic oligonucleotide
agagggcugggauaaucccuu

SEQ ID NO: 70 - RNA, 21 nt, Artificial Sequence
  Description of Artificial Sequence Synthetic oligonucleotide
aagagguggcuggucauauua

SEQ ID NO: 71 - RNA, 21 nt, Artificial Sequence
  Description of Artificial Sequence Synthetic oligonucleotide
uaauaugaccagccaccucuu

SEQ ID NO: 72 - RNA, 21 nt, Artificial Sequence
  Description of Artificial Sequence Synthetic oligonucleotide
aaaugaagagcaggacaaaag

SEQ ID NO: 73 - RNA, 21 nt, Artificial Sequence
  Description of Artificial Sequence Synthetic oligonucleotide
cuuuuguccugcucuucauuu

SEQ ID NO: 74 - RNA, 21 nt, Artificial Sequence
  Description of Artificial Sequence Synthetic oligonucleotide
aaauuggauacagaaagagac

SEQ ID NO: 75 - RNA, 21 nt, Artificial Sequence
  Description of Artificial Sequence Synthetic oligonucleotide
gucucuuucuguauccaauuu

SEQ ID NO: 76 - RNA, 21 nt, Artificial Sequence
  Description of Artificial Sequence Synthetic oligonucleotide
aaacacaacauggaacaauag

SEQ ID NO: 77 - RNA, 21 nt, Artificial Sequence
  Description of Artificial Sequence Synthetic oligonucleotide
cuauuguuccauguuguguuu

SEQ ID NO: 78 - RNA, 21 nt, Artificial Sequence
  Description of Artificial Sequence Synthetic oligonucleotide
aacauagaagcagaaccucca

SEQ ID NO: 79 - RNA, 21 nt, Artificial Sequence
  Description of Artificial Sequence Synthetic oligonucleotide
uggagguucugcuucuauguu

SEQ ID NO: 80 - RNA, 21 nt, Artificial Sequence
  Description of Artificial Sequence Synthetic oligonucleotide
aaagggaagacuguuugguuc

SEQ ID NO: 81 - RNA, 21 nt, Artificial Sequence
  Description of Artificial Sequence Synthetic oligonucleotide
gaaccaaacagucuucccuuu

SEQ ID NO: 82 - RNA, 21 nt, Artificial Sequence
  Description of Artificial Sequence Synthetic oligonucleotide
aaaaggaaaaguugugggucu

SEQ ID NO: 83 - RNA, 21 nt, Artificial Sequence
  Description of Artificial Sequence Synthetic oligonucleotide
agacccacaacuuuuccuuuu

SEQ ID NO: 84 - RNA, 21 nt, Artificial Sequence
  Description of Artificial Sequence Synthetic oligonucleotide
aauggccaucaguggagauga

SEQ ID NO: 85 - RNA, 21 nt, Artificial Sequence
  Description of Artificial Sequence Synthetic oligonucleotide
ucaucuccacugauggccauu

SEQ ID NO: 86 - RNA, 21 nt, Artificial Sequence
  Description of Artificial Sequence Synthetic oligonucleotide
aaaggugagaagcaaugcagc

SEQ ID NO: 87 - RNA, 21 nt, Artificial Sequence
  Description of Artificial Sequence Synthetic oligonucleotide
gcugcauugcuucucaccuuu

SEQ ID NO: 88 - RNA, 21 nt, Artificial Sequence
  Description of Artificial Sequence Synthetic oligonucleotide
aaaagcaagaaguaguugucc

SEQ ID NO: 89 - RNA, 21 nt, Artificial Sequence
  Description of Artificial Sequence Synthetic oligonucleotide
ggacaacuacuucuugcuuuu

SEQ ID NO: 90 - RNA, 21 nt, Artificial Sequence
  Description of Artificial Sequence Synthetic oligonucleotide
aaaauuggaauagguguccuc

SEQ ID NO: 91 - RNA, 21 nt, Artificial Sequence
  Description of Artificial Sequence Synthetic oligonucleotide
gaggacaccuauuccaauuuu

SEQ ID NO: 92 - RNA, 21 nt, Artificial Sequence
  Description of Artificial Sequence Synthetic oligonucleotide
aaaauccuuacaaaacguggg

SEQ ID NO: 93 - RNA, 21 nt, Artificial Sequence
  Description of Artificial Sequence Synthetic oligonucleotide
cccacguuuuguaaggauuuu

SEQ ID NO: 94 - RNA, 21 nt, Artificial Sequence
  Description of Artificial Sequence Synthetic oligonucleotide
aaauccuuacaaaacgugggc

SEQ ID NO: 95 - RNA, 21 nt, Artificial Sequence
  Description of Artificial Sequence Synthetic oligonucleotide
gcccacguuuuguaaggauuu

SEQ ID NO: 96 - DNA, 20 bp, Artificial Sequence
  Description of Artificial Sequence Synthetic oligonucleotide
ctatcaatatgctgaacgcg

SEQ ID NO: 97 - DNA, 19 bp, Artificial Sequence
  Description of Artificial Sequence Synthetic oligonucleotide
cggatgtggacttttcggg

SEQ ID NO: 98 - DNA, 20 bp, Artificial Sequence
  Description of Artificial Sequence Synthetic oligonucleotide
gacagaaggtggtgtttgat

SEQ ID NO: 99 - DNA, 19 bp, Artificial Sequence
  Description of Artificial Sequence Synthetic oligonucleotide
cagcatattgacacctggg

SEQ ID NO: 100 - DNA, 19 bp, Artificial Sequence
  Description of Artificial Sequence Synthetic oligonucleotide
ggactagaggttagaggag

SEQ ID NO: 101 - DNA, 19 bp, Artificial Sequence
  Description of Artificial Sequence Synthetic oligonucleotide
ggatgtggacttttcggga

SEQ ID NO: 102 - DNA, 19 bp, Artificial Sequence
  Description of Artificial Sequence Synthetic oligonucleotide
ggctgcggactgtttggaa

SEQ ID NO: 103 - DNA, 21 bp, Artificial Sequence
  Description of Artificial Sequence Synthetic oligonucleotide
gggagcattgacacatgtgca

SEQ ID NO: 104 - DNA, 19 bp, Artificial Sequence
  Description of Artificial Sequence Synthetic oligonucleotide
acacaacatggaacaatag

SEQ ID NO: 105 - DNA, 19 bp, Artificial Sequence
  Description of Artificial Sequence Synthetic oligonucleotide
catagaagcagaacctcca

SEQ ID NO: 106 - DNA, 21 bp, Artificial Sequence
  Description of Artificial Sequence Synthetic oligonucleotide
ggaacatcaggctcaccaata

SEQ ID NO: 107 - DNA, 22 bp, Artificial Sequence
  Description of Artificial Sequence Synthetic oligonucleotide
gggctttatggcaatggagtca

SEQ ID NO: 108 - DNA, 21 bp, Artificial Sequence
  Description of Artificial Sequence Synthetic oligonucleotide
tctgccacagatcatcaaaga

SEQ ID NO: 109 - DNA, 21 bp, Artificial Sequence
  Description of Artificial Sequence Synthetic oligonucleotide
gtggctgctgagatggctgaa

SEQ ID NO: 110 - DNA, 21 bp, Artificial Sequence
  Description of Artificial Sequence Synthetic oligonucleotide
ctcacccacaggctgatgtct

SEQ ID NO: 111 - DNA, 21 bp, Artificial Sequence
  Description of Artificial Sequence Synthetic oligonucleotide
gtgatggatgaggctcatttc

SEQ ID NO: 112 - DNA, 21 bp, Artificial Sequence
  Description of Artificial Sequence Synthetic oligonucleotide
gatacgaatggatcacagaat

SEQ ID NO: 113 - DNA, 21 bp, Artificial Sequence
  Description of Artificial Sequence Synthetic oligonucleotide
ggaagtcagagggtacacaaa

SEQ ID NO: 114 - DNA, 21 bp, Artificial Sequence
  Description of Artificial Sequence Synthetic oligonucleotide
ggtcaccatgaagagtggagt

SEQ ID NO: 115 - DNA, 21 bp, Artificial Sequence
  Description of Artificial Sequence Synthetic oligonucleotide
actccacgcacgagatgtgtt

SEQ ID NO: 116 - DNA, 21 bp, Artificial Sequence
  Description of Artificial Sequence Synthetic oligonucleotide
ccatggccatgactgacacta

SEQ ID NO: 117 - DNA, 21 bp, Artificial Sequence
  Description of Artificial Sequence Synthetic oligonucleotide
gccatttggttcatgtggctt

SEQ ID NO: 118 - DNA, 21 bp, Artificial Sequence
  Description of Artificial Sequence Synthetic oligonucleotide
tggacctggctgtttgagaat

SEQ ID NO: 119 - DNA, 21 bp, Artificial Sequence
  Description of Artificial Sequence Synthetic oligonucleotide
aatatcaaacaccaccaccga

SEQ ID NO: 120 - DNA, 21 bp, Artificial Sequence
  Description of Artificial Sequence Synthetic oligonucleotide
aaagctttgaaactaagctgg

SEQ ID NO: 121 - DNA, 21 bp, Artificial Sequence
  Description of Artificial Sequence Synthetic oligonucleotide
aagaagggcctctaccagaga

SEQ ID NO: 122 - DNA, 21 bp, Artificial Sequence
  Description of Artificial Sequence Synthetic oligonucleotide
aagggattatcccagccctct

SEQ ID NO: 123 - DNA, 21 bp, Artificial Sequence
  Description of Artificial Sequence Synthetic oligonucleotide
aagaggtggctggtcatatta

SEQ ID NO: 124 - DNA, 21 bp, Artificial Sequence
  Description of Artificial Sequence Synthetic oligonucleotide
aaatgaagagcaggacaaaag

SEQ ID NO: 125 - DNA, 21 bp, Artificial Sequence
  Description of Artificial Sequence Synthetic oligonucleotide
aaattggatacagaaagagac

SEQ ID NO: 126 - DNA, 21 bp, Artificial Sequence
  Description of Artificial Sequence Synthetic oligonucleotide
aaacacaacatggaacaatag

SEQ ID NO: 127 - DNA, 21 bp, Artificial Sequence
  Description of Artificial Sequence Synthetic oligonucleotide
aacatagaagcagaacctcca

SEQ ID NO: 128 - DNA, 21 bp, Artificial Sequence
  Description of Artificial Sequence Synthetic oligonucleotide
aaagggaagactgtttggttc

SEQ ID NO: 129 - DNA, 21 bp, Artificial Sequence
  Description of Artificial Sequence Synthetic oligonucleotide
aaaaggaaaagttgtgggtct

SEQ ID NO: 130 - DNA, 21 bp, Artificial Sequence
  Description of Artificial Sequence Synthetic oligonucleotide
aatggccatcagtggagatga

SEQ ID NO: 131 - DNA, 21 bp, Artificial Sequence
  Description of Artificial Sequence Synthetic oligonucleotide
aaaggtgagaagcaatgcagc

SEQ ID NO: 132 - DNA, 21 bp, Artificial Sequence
  Description of Artificial Sequence Synthetic oligonucleotide
aaaagcaagaagtagttgtcc

SEQ ID NO: 133 - DNA, 21 bp, Artificial Sequence
  Description of Artificial Sequence Synthetic oligonucleotide
aaaattggaataggtgtcctc

SEQ ID NO: 134 - DNA, 21 bp, Artificial Sequence
  Description of Artificial Sequence Synthetic oligonucleotide
aaaatccttacaaaacgtggg

SEQ ID NO: 135 - DNA, 21 bp, Artificial Sequence
  Description of Artificial Sequence Synthetic oligonucleotide
aaatccttacaaaacgtgggc

SEQ ID NO: 136 - protein, 10 aa, Artificial Sequence
  Description of Artificial Sequence Synthetic Kaposi fibroblast growth factor peptide
VQRKRQKLMP

SEQ ID NO: 137 - protein, 11 aa, Simian virus 40
SPKKKRKVEAS

SEQ ID NO: 138 - DNA, 23 bp, Artificial Sequence
  Description of Artificial Sequence Synthetic oligonucleotide
aannnnnnnnnnnnnnnnnnntt